SEQUENCE LISTING (37 sequences)

SEQ ID NO: 1 - protein, 501 aa, Artificial
  cam1615HER2
MEQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEDVQLTQSPAILSASPGEKVTMTCRATPSVSYMHWYQQKPGSSPKPWIYTTSNLASGVPARFSGGGSGTSYSLTVSRVEAEDAATYYCQQWSRSPPTFGGGSKLEIKGSTSGSGKSSEGKGVQLQESGPEVVKPGGSMKISCKTSGYSFTGHTMNWVKQSHGKNLEWIGLINPYNGDTNYNQKFKGKATFTVDKSSSTAYMELLSLTSEDSAVYYCARRVTDWYFDVWGAGTTVTVS

SEQ ID NO: 2 - protein, 122 aa, Artificial
  cam16
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 3 - protein, 20 aa, Artificial
  linker
PSGQAGAAASESLFVSNHAY

SEQ ID NO: 4 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 7 aa, Artificial
  linker
EASGGPE

SEQ ID NO: 6 - protein, 236 aa, Artificial
  anti-HER2
DVQLTQSPAILSASPGEKVTMTCRATPSVSYMHWYQQKPGSSPKPWIYTTSNLASGVPARFSGGGSGTSYSLTVSRVEAEDAATYYCQQWSRSPPTFGGGSKLEIKGSTSGSGKSSEGKGVQLQESGPEVVKPGGSMKISCKTSGYSFTGHTMNWVKQSHGKNLEWIGLINPYNGDTNYNQKFKGKATFTVDKSSSTAYMELLSLTSEDSAVYYCARRVTDWYFDVWGAGTTVTVS

SEQ ID NO: 7 - DNA, 1517 bp, Artificial
  cam1615HER2 DNA sequence
ccatggagcaggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctcaccgtctggtcaggctggtgctgctgctagcgaatctctgttcgtttctaaccacgcttacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaagcttccggaggtcccgaggacgtccagctgacccagtctccagcaatcctgtctgcatctccaggggagaaggtcacaatgacttgcagggccaccccaagtgtaagttacatgcactggtatcagcagaagccaggatcctcccccaaaccttggatttataccacatccaacctggcttctggagtccctgctcgcttcagtggcggtgggtctgggacctcttactctctcacagtcagcagagtggaggctgaagatgctgccacttattactgccagcagtggagtcgtagcccacccacgttcggaggggggtccaagctggaaataaaaggttctacctctggttctggtaaatcttctgaaggtaaaggtgtgcagctgcaggagtcaggacctgaggtggtgaagcctggaggttcaatgaagatatcctgcaagacttctggttactcattcactggccacaccatgaactgggtgaagcagagccatggaaagaaccttgagtggattggacttattaatccttacaatggtgatactaactacaaccagaagttcaagggcaaggccacatttactgtagacaagtcgtccagcacagcctacatggagctcctcagtctgacatctgaggactctgcagtctattactgtgcaaggagggttacggactggtacttcgatgtctggggcgcagggaccacggtcaccgtctcctaatagctcgag

SEQ ID NO: 8 - DNA, 8 bp, Artificial
  NcoI restriction site and start codon
ccatggag

SEQ ID NO: 9 - DNA, 366 bp, Artificial
  cam16
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctca

SEQ ID NO: 10 - DNA, 60 bp, Artificial
  linker
ccgtctggtcaggctggtgctgctgctagcgaatctctgttcgtttctaaccacgcttac

SEQ ID NO: 11 - DNA, 342 bp, Homo sapiens
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 12 - DNA, 21 bp, Artificial
  linker
gaagcttccggaggtcccgag

SEQ ID NO: 13 - DNA, 708 bp, Artificial
  anti-HER2
gacgtccagctgacccagtctccagcaatcctgtctgcatctccaggggagaaggtcacaatgacttgcagggccaccccaagtgtaagttacatgcactggtatcagcagaagccaggatcctcccccaaaccttggatttataccacatccaacctggcttctggagtccctgctcgcttcagtggcggtgggtctgggacctcttactctctcacagtcagcagagtggaggctgaagatgctgccacttattactgccagcagtggagtcgtagcccacccacgttcggaggggggtccaagctggaaataaaaggttctacctctggttctggtaaatcttctgaaggtaaaggtgtgcagctgcaggagtcaggacctgaggtggtgaagcctggaggttcaatgaagatatcctgcaagacttctggttactcattcactggccacaccatgaactgggtgaagcagagccatggaaagaaccttgagtggattggacttattaatccttacaatggtgatactaactacaaccagaagttcaagggcaaggccacatttactgtagacaagtcgtccagcacagcctacatggagctcctcagtctgacatctgaggactctgcagtctattactgtgcaaggagggttacggactggtacttcgatgtctggggcgcagggaccacggtcaccgtctcc

SEQ ID NO: 14 - DNA, 13 bp, Artificial
  two stop codons and XhoI restriction site
taatagctcgaga

SEQ ID NO: 15 - protein, 236 aa, Artificial; identical to SEQ ID NO: 6
  e23

SEQ ID NO: 16 - protein, 248 aa, Artificial
  trastuzumab-based scFv
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 17 - protein, 214 aa, Artificial
  anti-HER2 light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 225 aa, Artificial
  anti-HER2 heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 19 - protein, 449 aa, Artificial
  lumretuzumab heavy chain
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 220 aa, Artificial
  lumretuzumab light chain
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 445 aa, Artificial
  seribantumab heavy chain
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 217 aa, Artificial
  seribantumab light chain
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 23 - protein, 216 aa, Artificial
  KTN3379 light chain
QSVLTQPPSASGTPGQRVTISCSGSLSNIGLNYVSWYQQLPGTAPKLLISRNNQRPSGVPDRFSGSKSGTSASLAISGLRSEDEADYYCAAWDDSPPGEAFGGGTKLTVLGQPKAAPSVTLFPPSSEELQANKATLVCLISDFYPGAVTVAWKADSSPVKAGVETTKPSKQSNNKYAASSYLSLTPEQWKSHRSYSCQVTHEGSTVEKTVAPTECS

SEQ ID NO: 24 - protein, 447 aa, Artificial
  patritumab subunit 1
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 447 aa, Artificial; identical to SEQ ID NO: 24
  patritumab subunit 2

SEQ ID NO: 26 - protein, 220 aa, Artificial
  patritumab subunit 3
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 220 aa, Artificial; identical to SEQ ID NO: 26
  patritumab subunit 4

SEQ ID NO: 28 - protein, 524 aa, Artificial
  trastuzumab-based TriKE
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 29 - DNA, 1572 bp, Artificial
  trastuzumab-based TriKE, H. sapiens codon optimized
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctcatctggcggcggcggttctggtggaggaggtagtggggggggaggaagcggagggggtggctcagggaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggcagtaccagcgggtcagggaaacctggcagtggggaaggttccacaaaaggtgaggttcagctcgtggaatccggcggcgggctggtccaaccaggtgggagtctccgcctgtcatgtgccgcaagcggattcaatataaaagatacatatatacattgggtaagacaggcccccggtaagggtctggagtgggttgccagaatttatcccactaatggatacactcgttacgccgattctgtgaaaggccggtttaccatctctgctgacacctcaaagaacaccgcgtacctccagatgaactccctgagggcagaggacacggctgtctattattgctctcgctggggcggcgacgggttttatgccatggattactggggtcaggggaccctagttacagtgagcagcggtagtacttctgggagcggcaagcctggctccggagaaggatccaccaaaggggatatccagatgacgcagagcccatcatcattgagtgccagtgtgggcgaccgggtcacgatcacctgtagggcatctcaggacgtaaacacagcggtggcatggtaccagcaaaaacctggaaaggccccaaaacttttgatctacagcgctagcttcttatactccggcgtcccctcacgattctccggctccagaagtggaacagactttactctgacaatttcttcgcttcagcccgaggattttgctacctattattgccagcaacactacacaacccctccgactttcggacaagggacaaaggtggaaattaagaggactgtg

SEQ ID NO: 30 - DNA, 1572 bp, Artificial
  trastuzumab-based TriKE, E. coli codon optimized
caggtgcagctggtggagtctggtggcggcttggtgcagcctggtggctctctgcgcctgtcctgtgcggcctctggcctcaccttcagcagctataacatgggctggttccgccaggctccaggacaaggccttgaggctgtggcgtctattacctggagtggtcgggacaccttctatgcggactccgtgaaaggccgtttcaccatctcgcgtgacaattccaagaacacgctgtatctgcaaatgaacagcctgcgcgcggaggacacggccgtttattattgtgcggcaaacccctggccggttgcggcgccgcgtagtggcacctactggggccaagggaccctggtcaccgtctcctcatctggcggcggcggtagcggtggcggaggtagcggggggggtggaagcggtggtggtggctcagggaactgggtgaatgtaataagtgatttgaaaaaaattgaagatctgattcagagcatgcatattgatgcgacgttatatacggaatcggatgttcacccaagttgcaaagttacagcaatgaaatgctttttgttagagttacaagttatttcacttgagtcgggagatgcaagtattcatgatactgtagaaaatctgatcatcctggcaaacaacagcttgtcgtcgaatgggaatgtaacagaatctggatgcaaagaatgtgaagaactggaagaaaaaaatattaaagaatttttgcagagttttgttcacattgtccaaatgttcatcaacacttctggcagtaccagcggttcaggtaaaccgggcagcggggaaggttccacaaaaggtgaagttcagctcgtggaaagcggcggcggtctggtccagccaggtgggagtctccgcctgtcatgtgccgcgagcggttttaatatcaaagatacatatatacattgggtaagacaggccccgggtaagggtctggagtgggttgcgcgtatttatccgacgaatggatacactcgttacgccgattctgtgaaaggccgctttaccatctcagccgacacctcaaagaacaccgcgtacttacagatgaactccctgcgcgcagaagacacggctgtctattattgctcgcgctggggcggcgacggtttttatgccatggattactggggtcaggggaccctagttactgtgagcagcggtagtacttctgggagcggcaaacctggctccggagaaggttcgaccaaaggggatatccagatgacgcagagcccgtcatcactgtcggccagtgtgggcgatcgggtcacgatcacctgccgtgcatcgcaggatgtaaatacagcggtggcatggtaccagcaaaaacctggaaaggccccaaaacttctgatctacagcgctagcttcttatactccggcgtcccgtcacgattttccggctcccgtagtggaacggactttactctgacaatttcttcgcttcagcccgaggattttgctacctattattgccagcaacactacaccaccccgccgactttcggccaagggacgaaagtggaaattaagaggacggtg

SEQ ID NO: 31 - protein, 497 aa, Artificial
  partial trastuzumab-based TriKE
MGWSCIILFLVATATGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 32 - protein, 244 aa, Artificial
  partial trastuzumab-based TriKE
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 33 - protein, 759 aa, Artificial
  trastuzumab-based TriKE
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKEGRGSLLTCGDVEENPGPMGWSCIILFLVATATGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 34 - protein, 22 aa, Artificial
  linker
SGGGGSGGGGSGGGGSGGGGSG

SEQ ID NO: 35 - protein, 18 aa, Artificial
  linker
GSTSGSGKPGSGEGSTKG

SEQ ID NO: 36 - protein, 19 aa, Artificial
  signal peptide
MGWSCIILFLVATATGVHS

SEQ ID NO: 37 - protein, 18 aa, Artificial
  T2A self-cleaving peptide
EGRGSLLTCGDVEENPGP

(19) United States
(12) Patent Application Publication
(10) Pub. No.: US 2022/0324972 A1
(43) Pub. Date: Oct. 13, 2022

(54) IMMUNOTHERAPY COMPOUNDS AND METHODS

(71) Applicants: REGENTS OF THE UNIVERSITY OF MINNESOTA, Minneapolis, MN (US); GT BIOPHARMA, INC., Brisbane, CA (US)

(72) Inventors: Daniel A. VALLERA, Richfield, MN (US); Jeffrey S. MILLER, North Oaks, MN (US); Martin FELICES, Circle Pines, MN (US); Martin SCHROEDER, San Rafael, CA (US)

(21) Appl. No.: 17/641,594

(22) PCT Filed: Sep. 15, 2020
(86) PCT No.: PCT/US2020/050851

§ 371 Date: Mar. 9, 2022

Related U.S. Application Data
(60) Provisional application No. 62/901,198, filed on Sep. 16, 2019.

(51) Int. Cl. C07K 16/28; C07K 14/54

(52) U.S. Cl. CPC C07K 16/283; C07K 14/5443; A61K 2039/505

(57) ABSTRACT

An immunotherapy compound includes an NK cell engaging domain, an NK activating domain and a targeting domain. The targeting domain selectively binds to HER2, HERS, or the HER2/HER3 heterodimer complex and is operably linked to the NK activating domain and the NK cell engaging domain. The compound may be administered to a subject to induce NK-mediated killing of a cancer cell, to stimulate expansion of NK cells in the subject, and/or for treating cancer.

### CROSS-REFERENCE TO RELATED APPLICATION

This application claims the benefit of U.S. Provisional Patent Application No. 62/901,198, filed Sep. 16, 2019, which is incorporated herein by reference in its entirety.

### GOVERNMENT FUNDING

This invention was made with government support under CA197292 awarded by the National Institutes of Health. The government has certain rights in the invention.

### SEQUENCE LISTING

This application contains a Sequence Listing electronically submitted via EFS-Web to the United States Patent and Trademark Office as an ASCII text file entitled “Seq_Listing-0110-000632_ST25.txt” having a size of 70 kilobytes and created on Sep. 15, 2020. The information contained in the Sequence Listing is incorporated by reference herein.

### SUMMARY

This disclosure describes, in one aspect, a multispecific immunotherapeutic compound that includes an NK cell engaging domain, an NK activating domain and a targeting domain. The targeting domain selectively binds to HER2, HER3, or the HER2/IER3 heterodimer complex and is operably linked to the NK activating domain and the NK cell engaging domain.

In some embodiments, the NK cell engaging domain specifically binds to CD16. In these embodiments, the CD16 can be CD16a or CD16b. In some of these embodiments the NK cell engaging domain includes the amino acid sequence of SEQ ID NO:2.

In some embodiments the NK cell engaging domain moiety can include an antibody or a binding fragment thereof. In some of these embodiments, the antibody or a binding fragment thereof can be human, humanized, or camelid.

In some embodiments, the NK activating domain includes an IL-15 component. In some of these embodiments, the IL-15 component includes the amino acid sequence of SEQ ID NO.4 or a functional variant thereof. In some of these embodiments, the functional variant of IL-15 includes an N72D or N72A amino acid substitution as compared to SEQ ID NO:4.

In some embodiments, the targeting domain includes an antibody or a binding fragment thereof. In some of these embodiments, the antibody binding fragment can include an scFv, a F(ab)2, a Fab, or a single-domain antibody fragment. In some of these embodiments, the targeting domain includes the amino acid sequence of SEQ ID NO:6, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, or SEQ ID NO:27.

In some embodiments, the immunotherapeutic compound can include a second targeting domain.

In some embodiments, the immunotherapeutic compound can include a second NK cell engaging domain.

In some embodiments, the immunotherapeutic compound can include a second NK activating domain.

In another aspect, this disclosure describes a composition that includes any embodiment of the therapeutic compound summarized above and a pharmaceutically acceptable carrier.

In some embodiments, the composition can further include an additional therapeutic agent. In some of these embodiments, the additional therapeutic agent can include a chemotherapeutic agent. In some embodiments, the additional therapeutic agent can include a therapeutic agent that targets HER2, HER3, or the HER2/IER3 heterodimer complex.

In another aspect, this disclosure describes a method that includes administering to a subject any embodiment of the compound or composition summarized above in an amount effective to induce NK-mediated killing of a cancer cell.

In another aspect, this disclosure describes a method for stimulating expansion of NK cells in vivo. Generally, the method includes administering to a subject an amount of any embodiment of the compound or composition summarized above effective to stimulate expansion of NK cells in the subject.

In another aspect, this disclosure describes a method of treating cancer in a subject. Generally, the method includes administering to the subject an amount of any embodiment of the compound or composition summarized above effective for treating the cancer.

In some embodiments, the compound or composition is administered prior to, simultaneously with, or following chemotherapy, surgical resection of a tumor, or radiation therapy. In some of these embodiments, the chemotherapy can include altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, or vinorelbine.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Design, production, and purification of cam1615HER2. (A) Schematic illustration of an exemplary expression vector with arrangement of coding regions that encode components of cam1615HER2 (left to right): camelid anti-CD16 VHH, human IL-15, anti-HER2 scFV. (B) Gene map of the exemplary expression vector encoding cam1615HER2, including restriction sites and target gene positions on the pET28c vector.

FIG. 2. Production and purification of cam1615HER2. (A) SDS-PAGE gel stained with Coomassie Blue dye, indicating the purity and size of the final product after the two orthogonal column steps. MWS: molecular weight standards; NR: nonreduced; R: Reduced. Densitometry was performed to derive the final purity. (B) Chromatography trace resulting from the first-step purification of cam1615HER2 on an ion exchange (FFQ) column. The collection peak is indicated by the double-sided arrow. (C) Chromatography trace of resulting from the second-step purification of cam1615HER2 on size exclusion column. The collection peak is indicated by the double-sided arrow.

FIG. 3. cam1615HER2 effects on NK cell expansion and absolute numbers showing the effect of TriKE treatment on the percentage of C56⁺CD3⁻ NK cells undergoing proliferation and measured by flow cytometry. PBMCs from six different normal donors were separately assayed. In addition to cam1615HER2, IL-15 was used as a control. (A) cam1615HER2 treatment shows percentage of highly proliferating NK cells were significantly different when compared to the controls. (B) cam1615HER2 treatment shows percentage of total NK cells were significantly different when compared to the controls. (C) cam1615HER2 treatment shows numbers of raw NK counts were significantly different when compared to the NT control. (D) cam1615HER2 treatment did not increase percentage of highly proliferating CD3⁺CD56⁻ T cells. (E) cam1615HER2 treatment did not increase percentage of total CD3⁺CD56⁻ T cells. (F) cam1615HER2 treatment did not increase numbers of raw CD3⁺CD56⁻ T cell.

FIG. 4. cam1615HER2 TriKE binding to target cell lines. (A) cam1615HER2 directly labeled with FITC binding to SKOV-3 cell line. (B)) cam1615HER2 directly labeled with FITC binding to SK-BR-3 cell line. (C)) cam1615HER2 directly labeled with FITC binding to UMSCC-11B cell line. BAC3 is a CD3 binding molecule and serves as a negative control for binding.

FIG. 5. Functional activity correlates with binding activity of cam1615HER2 TriKE. (A) CD107a functional activity was elevated in cultures of PBMCs plus SKOV3 cells treated with cam1615HER2 compared to IL-15 and no treatment controls. PBMC from 10 different normal donors separately assayed. (B) Enhanced IFN-γ activity in the same PBMC/SKOV3 cultures (C) CD107a functional activity was elevated in cultures of PBMCs plus breast cancer cell line SK-BR-3 (seven different donors assayed). (D) Enhanced IFN-γ activity in the same PBMC/SK-BR-3 cultures. (E) CD107a was not elevated when the UMSCC-11B head and neck cancer cell line was tested in the same assay (four different donors assayed).

FIG. 6. Testing of the ability of TriKE to augment the killing of drug (tamoxifen) resistant MCF-7L-TamR breast carcinoma cells. (A) Background cytotoxic activity of CD56⁺CD3⁻ NK cells without cancer cells was tested using CD107a flow cytometry. (B) CD107a activity using PBMCs incubated with the parental MCF-7L cell line. (C) CD107a activity using PBMCs incubated with the tamoxifen resistant MCF-7L-TamR cells. (D) CD107a activity using PBMCs incubated with SKBR-3 breast cancer cells. (E) Background intracellular IFN-γ activity of CD56⁺CD3⁻ NK cells without cancer cells. (F) Intracellular IFN-γ activity using PBMCs incubated with the parental MCF-7L cell line. (G) Intracellular IFN-γ activity using PBMCs incubated with the tamoxifen resistant MCF-7L-TamR cells. (H) Intracellular IFN-γ activity using PBMCs incubated with SKBR-3 breast cancer cells. Intracellular IFN-γ activity correlated with CD107a activity.

FIG. 7. INCUCYTE (Essen Bioscience, Inc., Ann Arbor, Mich.) data measuring killing in real time of SKOV3 ovarian cancer cells in the presence of PBMCs, confirming CD107a cytotoxicity data. (A) Spheroid size; (B) spheroid intensity. Cam1615HER2 causes a precipitous decline in target cells measured over a 120-hour period compared to lesser activity in no treatment, anti-cam16 alone (CAM16), and IL-15 alone (IL15) controls. N=7 donors/group.

FIG. 8. Visual evidence that cam1615HER2 causes a precipitous time-dependent decline in target cells measured over a 72-hour period (right column) compared to lesser activity in no treatment (left column), anti-cam16 alone, and IL-15 alone controls. N=7 donors/group.

FIG. 9. Testing ascites from ovarian cancer patients as a source of effector cells. (A) CD107a background activity when cells from patient ascites fluid were incubated without MA-148 ovarian cancer cells. (B) CD107a activity when ascites cells were incubated with MA-148 cells ovarian cancer cells. (C) CD107a background activity when cells from normal donors were incubated without MA-148 ovarian cancer cells. (D) CD107a activity when normal donor cells were incubated with MA-148 cells ovarian cancer cells. (E) IFN-γ background activity when cells from patient ascites fluid were incubated without MA-148 ovarian cancer cells. (F) IFN-γ activity when ascites cells were incubated with MA-148 cells ovarian cancer cells. (G) IFN-γ background activity when cells from normal donors were incubated without MA-148 ovarian cancer cells. (H) IFN-γ activity when normal donor cells were incubated with MA-148 cells ovarian cancer cells. Controls were IL-15 and no treatment. In each case, 9-13 different donors were independently assayed and data averaged.

FIG. 10. In vivo efficacy of cam1615HER2 in a xenograft model. The cells were stably transfected with firefly luciferase for purposes of real time bioluminescent imaging. (A) Bioluminescent imaging of a group of six NSG mice given SKOV3 and NK cells intraperitoneally. The image shows total flux for each animal and indicates that five of six animals in the no treatment group had advanced tumor. The single animal in the no treatment group that showed minimal activity went on to develop tumor. (B) Day 38 imaging of a group of six mice also given SKOV3 and NK cells, but treated with cam1615HER2 TriKE.

FIG. 11. (A) Despite multiple TriKE injections, there were minimal changes in animal weight, indicating that treatment was not toxic compared to no treatment controls. (B) A scatter plot of data from the same experiment on Day 46 post-tumor inoculation. Data are expressed as Total flux Radiance (p/s). Treated mice are compared to untreated mice. The differences are significant as determined by Student T test (p=0.0216). (C) Line plot in time (days) indicating that the treated group has begun to relapse. (d) Survival plot of data over an extended time interval. Difference in treated versus untreated groups are significant.

FIG. 12. Testing of the ability of cam1615HER2 TriKE to augment the killing of other HER2 expressing ovarian cancer cell lines. (A) CD107a activity when PBMC NK cells were incubated with OVCAR3 ovarian cancer cells. (B) CD107a activity when PBMC NK cells were incubated with OVCAR5 ovarian cancer cells. (C) CD107a activity when PBMC NK cells were incubated with SKOV3 ovarian cancer cells. (D) IFN-γ activity when PBMC NK cells were incubated with OVCAR3 ovarian cancer cells. (E) IFN-γ activity when PBMC NK cells were incubated with OVCAR5 ovarian cancer cells. (F) IFN-γ activity when PBMC NK cells were incubated with SKOV3 ovarian cancer cells. The SKOV3 data were performed with the following negative controls: no treatment (NT), anti-cam16 alone (CAM16), IL-15 (IL15), and anti-HER2 antibody alone (e23).

### DETAILED DESCRIPTION OF ILLUSTRATIVE EMBODIMENTS

This disclosure generally describes therapeutic compounds that target tumor cells that express human epidermal growth factor receptor-2 (HER2) and or human epidermal growth factor receptor-3 (HER3), members of the epidermal growth factor receptor (EGFR) family of transmembrane receptor tyrosine kinases. HER2 has a direct association with cancer since its overexpression is associated with poor prognosis in breast cancer and it triggers intracellular signaling pathways related to cell proliferation, differentiation, and survival Her2 and HER3 can form a heterodimeric complex.

In many embodiments, the immunotherapeutic compound can be a Trispecific Killer Engager compound (TriKEs). TriKEs have three separate binding regions: an NK cell engaging domain that binds to an NK cell (e.g., CD16), an NK activating domain that includes a cytokine or a functional fragment thereof that binds to a receptor for that cytokine, and a targeting domain that binds a marker present on a target cell (e.g., a cancer cell). The design and production of TriKEs are broadly described in, for example, U.S. Patent Application Publication No. US 2018/0282386 A1. TriKEs offer the advantage of combining an antibody-dependent cellular cytotoxicity (ADCC)-facilitating moiety and an expansion-related moiety (IL-15) on the same molecule.

One or more of the binding regions or domains in the immunotherapeutic compound can include an antibody. As used herein, the term “antibody” refers generally an immunoglobulin or a fragment thereof and thus encompasses a monoclonal antibody, a fragment thereof. Exemplary antibody fragments include, but are not limited to, an scFv, a Fab, a F(ab′)₂, an Fv, a single-domain Ab (sdAb), or other modified forms (e.g., humanized), and/or a combination of monoclonal antibodies and/or fragments thereof. For example, camelids produce functional antibodies devoid of light chains. These single-domain antibody fragments (VHHs or NANOBODIES (Ablynx N.V., Ghent, Belgium)) have several advantages for biotechnological applications. They are well expressed in microorganisms and have a high stability and solubility. In certain embodiments of the TriKE compounds described herein, the NK cell engaging domain is a camelid single-domain antibody fragment.

While described herein in the context of an exemplary embodiment in which the immunotherapeutic compound has a targeting domain that includes a HER2-targeting scFv having the amino acid sequence of SEQ ID NO:6, the immunotherapeutic compounds described herein can include any other suitable HER2-targeting and or HER3-targeting moiety. Thus, in various embodiments, the targeting domain can recognize HER2, HER3, and/or the HER2/HER3 heterodimer. HER2/HER3 heterodimers are detected in many breast cancers and many HER2⁺ tumors. HER2/HER3 dimers are associated with proliferation, distant metastasis, and/or poor patient outcome.

Exemplary alternative targeting moieties include, antibodies and antibody fragments that specifically bind to HER2, HER3, and/or the HER2/IER3 heterodimer. Exemplary antibody fragments include but are not limited to, e23 or a functional fragment thereof (e.g., SEQ ID NO:15), trastuzumab or a functional fragment thereof (e.g., SEQ ID NO:16 and European Patent No. EP 3457139 A1), SEQ ID NO:17, SEQ ID NO:18, lumretuzumab or a functional fragment thereof (RG7116; Liu et al., 2019, BiolProced Online 21:5; e.g., SEQ ID NO:19, SEQ ID NO:20), seribantumab or a functional fragment thereof (MM-121; Liu et al., 2019, BiolProced Online 21:5; e.g., SEQ ID NO:21 or SEQ ID NO:22), KTN3379/CDX-3379 or a functional fragment thereof (Liu et al., 2019, BiolProced Online 21:5; e.g., SEQ ID NO:23), patritumab or a functional fragment thereof (U3-1287; Liu et al., 2019, Biol Proced Online 21:5; e.g., SEQ ID NO:24, SEQ ID NO:25, SEQ ID NO:26, or SEQ ID NO:27), elgemtumab (LJM716, Liu et al., 2019, Biol Proced Online 21:5) or a functional fragment thereof, U3-1402 (Liu et al., 2019, Biol Proced Online 21:5) or a functional fragment thereof, AV-203 (Liu et al., 2019, BiolProced Online 21:5) or a functional fragment thereof, GSK2849330 (Liu et al., 2019, BiolProced Online 21:5) or a functional fragment thereof, MM-111 (Liu et al., 2019, Biol Proced Online 21:5) or a functional fragment thereof, MCLA-128 (Liu et al., 2019, BiolProced Online 21:5) or a functional fragment thereof, istiratumab (MM-141; Liu et al., 2019, BiolProced Online 21:5) or a functional fragment thereof, duligotumab (MEHD7945A; Liu et al., 2019, BiolProced Online 21:5) or a functional fragment thereof, or pertuzumab or a functional variant thereof.

While described herein in the context of an exemplary embodiment in which the NK cell engaging domain includes a single-domain antibody (sdAb) that binds to CD16, an immunotherapeutic compound can include any other suitable NK engaging moiety. Exemplary alternative NK engaging moieties include, but are not limited to, any amino acid sequence that can selectively bind to a receptor at least partially located at the surface of an NK cell. Thus, the NK cell engaging domain can serve a function of binding an NK cell and thereby bring the NK into spatial proximity with a target to which the targeting domain selectively binds. In certain embodiments, the NK cell engaging domain can selectively bind to a receptor that activates the NK cell and, therefore, also possess an activating function. For example, activation of the CD16 receptor can elicit antibody-dependent cell-mediated cytotoxicity. Thus, the NK cell engaging domain of the exemplary cam1615HER2 compound possesses NK activating activity. In other embodiments, the NK cell engaging domain may interrupt mechanisms that inhibit NK cells. In such embodiments, the NK cell engaging domain can include, for example, anti-PD-1/PD-L1, anti-NKG2A, anti-TIGIT, anti-killer-immunoglobulin receptor (KIR), and/or any other inhibition blocking domain.

The NK cell engaging domain can include an antibody or ligand that selectively binds to any NK cell receptor such as, for example, the cell cytotoxicity receptor 2B4, low affinity Fc receptor CD16, killer immunoglobulin like receptors (KIR), CD2, NKG2A, TIGIT, NKG2C, LIR-1, and/or DNAM-1.

One can design the NK cell engaging domain to possess a desired degree of NK selectivity and, therefore, a desired immune engaging character. For example, CD16 has been identified as Fc receptors FcγRIIIa (CD16a) and FcγRIIIb (CD16b). These receptors bind to the Fc portion of IgG antibodies that then activate the NK cell for antibody-dependent cell-mediated cytotoxicity. Anti-CD16 antibodies selectively bind to NK cells, but also can bind to neutrophils. Anti-CD16a antibodies selectively bind to NK cells, but do not bind to neutrophils. An immunotherapeutic compound that includes an NK cell engaging domain having an anti-CD16a antibody can bind to NK cells but not bind to neutrophils. Thus, in circumstances where one may want to engage NK cells but not engage neutrophils, one can design the NK cell engaging domain of the immunotherapeutic compound to include an anti-CD16a antibody.

While described herein in the context of an exemplary embodiment in which the NK activating domain includes a fragment of human IL-15, the NK activating domain can include any amino acid sequence that activates NK cells, promotes sustaining NK cells, or otherwise promotes NK cell activity. The NK activating domain can be, or can be derived from, one or more cytokines that can activate and/or sustain NK cells. As used herein, the term “derived from” refers to an amino acid fragment of a cytokine (e.g., IL-15) that is a functional variant of the referenced cytokine—i.e., has sufficient sequence similarity or sequence identity to the referenced cytokine to provide NK cell activating and/or sustaining activity. Exemplary cytokines on which an NK activating domain may be based include, for example, IL-15, IL-18, IL-12, and IL-21. Thus, while the exemplary cam1615HER2 compound includes an NK activating domain derived from IL-15, a HER2-targeting compound may be designed to have an NK activating domain that is, or is derived from, any suitable cytokine.

For brevity in this description, reference to an NK activating domain by identifying the cytokine on which it is based can refer to either the full amino acid sequence of the cytokine or a functional variant of the cytokine. A functional variant of the cytokine can include any suitable amino acid fragment of the cytokine and/or a modified version of the cytokine that includes one or more amino acid deletions, additions, and/or substitutions. Thus, reference to an “IL-15” NK activating domain includes an NK activating domain that includes the full amino acid sequence of IL-15, an NK activating domain that includes a fragment of IL-15, or an NK activating domain such as, for example, IL-15N72D or IL-15N72A, that includes an amino acid substitution compared to the wild-type IL-15 amino acid sequence.

While described above in the context of an exemplary embodiment in which the immunotherapeutic compound is a trispecific Killer engager compound (i.e., a TriKE), the compositions and methods described herein can involve the use of immunotherapeutic compound modified to include additional domains. For example, the immunotherapeutic compound can be designed to be a larger molecule, having more than one targeting domain, more than one NK cell engaging domain, and/or more than one NK activating domain. In embodiments that include more than one NK activating domain, the NK activating domains may be provided in series or in any other combination. Any cytokine-based NK activating domain can include either the full amino acid sequence of the cytokine, may be an amino acid fragment, or may be a modified version of the cytokine, independent of the nature of other NK activating domains included in the immunotherapeutic compound.

Exemplary additional targeting domains include, but are not limited to, any moiety that selectively binds to an intended target such as, for example, a tumor cell, a target in the cancer stroma, a target on an inhibitory cell such as myeloid derived suppressor cells that are CD33+, or a target on a virally-infected cell. Thus, a targeting domain can include, for example, an anti-tumor antibody such as rituximab (anti-CD20), afutuzumab (anti-CD20), pertuzumab (anti-HER2/neu), labetuzumab (anti-CEA), adecatumumab (anti-EpCAM), citatuzumab bogatox (anti-EpCAM), edrecolomab (anti-EpCAM), arcitumomab (anti-CEA), bevacizumab (anti-VEGF-A), cetuximab (anti-EGFR), nimotuzumab (anti-EGFR), panitumumab (anti-EGFR), zalutumumab (anti-EGFR), gemtuzumab ozogamicin (anti-CD33), lintuzumab (anti-CD33), etaracizumab (anti-integrin aᵥβ₃), intetumumab (anti-CD51), ipilimumab (anti-CD152), oregovomab (anti-CA-125), votumumab (anti-tumor antigen CTAA16.88), or pemtumumab (anti-MUC1), anti-CD19, anti-CD20, anti-CD22, anti-CD23, anti-CD30, anti-CD38, anti-CD45, anti-CD52, anti-CD70, anti-CD74, anti-CD133, anti-mesothelin, anti-ROR1, anti-CSPG4, anti-SS1, or anti-HSPG2, anti-IGF-1, anti-ROR-1, anti-uPAR, anti-VEGFR, anti-LIV-1, anti-SGN-CD70A, anti-IL-3, anti-IL-4R, anti-epithelial-mesenchymal transition (EMT), anti-TRAIL, anti-PD-L1, any one or more of SEQ ID NOs:1-119 of European Patent No. EP 3457139 A1, or a functional variant of any of the foregoing.

An amino acid sequence is a “functional variant” of a reference amino acid sequence if the “functional variant” amino acid sequence possesses a specified amount of sequence identity or sequence specificity compared to the reference amino acid sequence. An amino acid sequence is a “functional fragment” of a reference amino acid sequence if the “functional fragment” amino acid sequence contains less than the full-length amino acid sequence of the reference amino acid sequence. A “functional fragment” may further possess a specified amount of sequence identity or sequence specificity compared to the reference amino acid sequence.

Sequence similarity and/or sequence identity of two amino acid sequences can be determined by aligning the residues of the amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order.

A pair-wise comparison analysis of amino acid sequences can be carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but allows for the presence of conservative substitutions. A conservative substitution for an amino acid residue within an amino acid sequence may be selected from other members of the class to which the amino acid residue belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Thus, conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂.

Thus, an amino acid belonging to a grouping of amino acids having a particular size or characteristic (e.g., charge, hydrophobicity, or hydrophilicity) can be substituted for another amino acid without altering the activity of a protein, particularly in regions of the protein that are not directly associated with biological activity. Regions within an amino acid sequence that are not directly associated with biological activity may be deduced from alignment analyses, identifying regions where variability (e.g., additions, deletions, or non-conservative substitutions) are present when comparing related amino acid sequences. One can perform the alignment analysis using amino acid sequences provided herein and/or amino acid sequences readily available in databases.

An NK engaging domain, an NK activating domain, or a targeting domain can include an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to a reference amino acid sequence (e.g., a reference antibody fragment, cytokine, or cytokine fragment).

An NK engaging domain, an NK activating domain, or a targeting domain can include an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

An immunotherapeutic compound as described herein also can be designed to provide additional sequences, such as, for example, the addition of added C-terminal or N-terminal amino acids that can, for example, facilitate purification by trapping on columns or use of antibodies. Such tags include, for example, histidine-rich tags that allow purification of polypeptides on nickel columns. Such gene modification techniques and suitable additional sequences are well known in the molecular biology arts.

This disclosure also provides polynucleotides that encode any of the immunotherapeutic compounds described herein, and the complements of such polynucleotide sequences. Given the amino acid sequence of any one of the immunotherapeutic compounds polypeptides described herein (or one or more component fragments of the immunotherapeutic compound), a person of ordinary skill in the art can determine the full scope of polynucleotides that encode that amino acid sequence using conventional, routine methods.

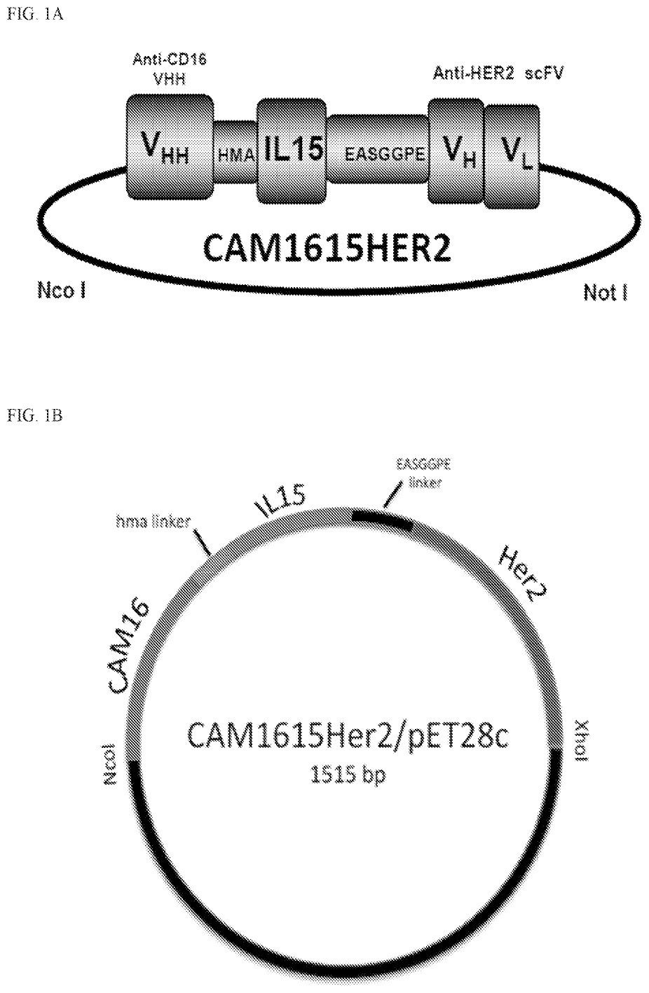

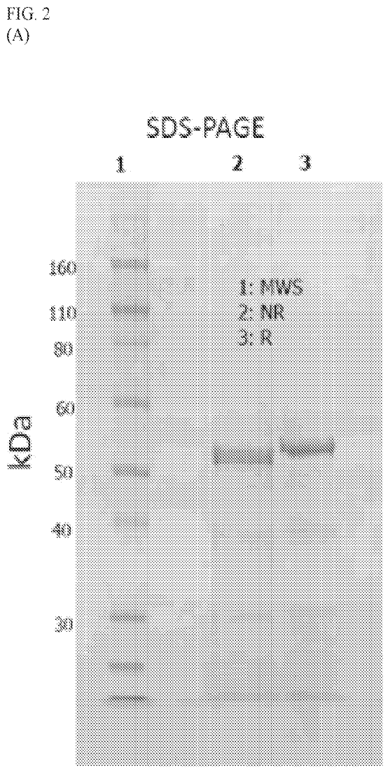

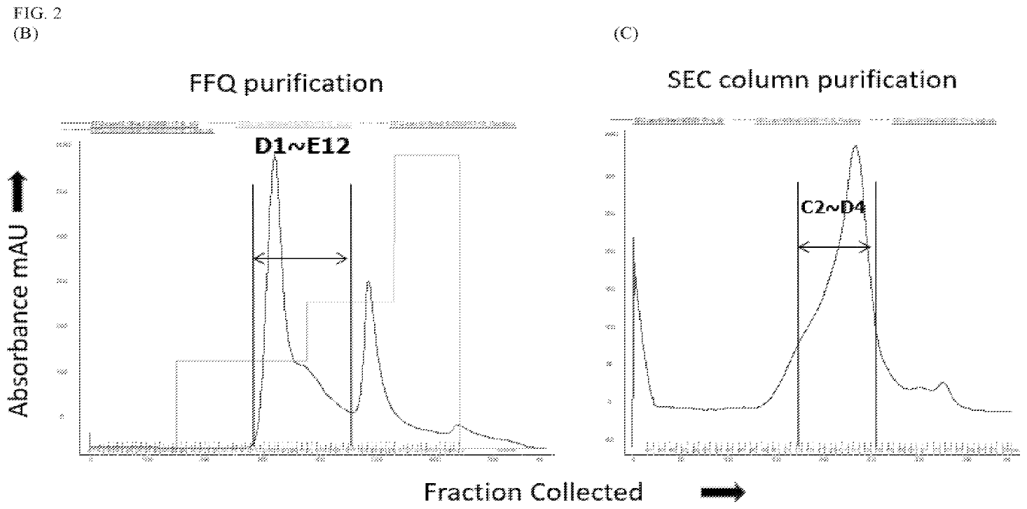

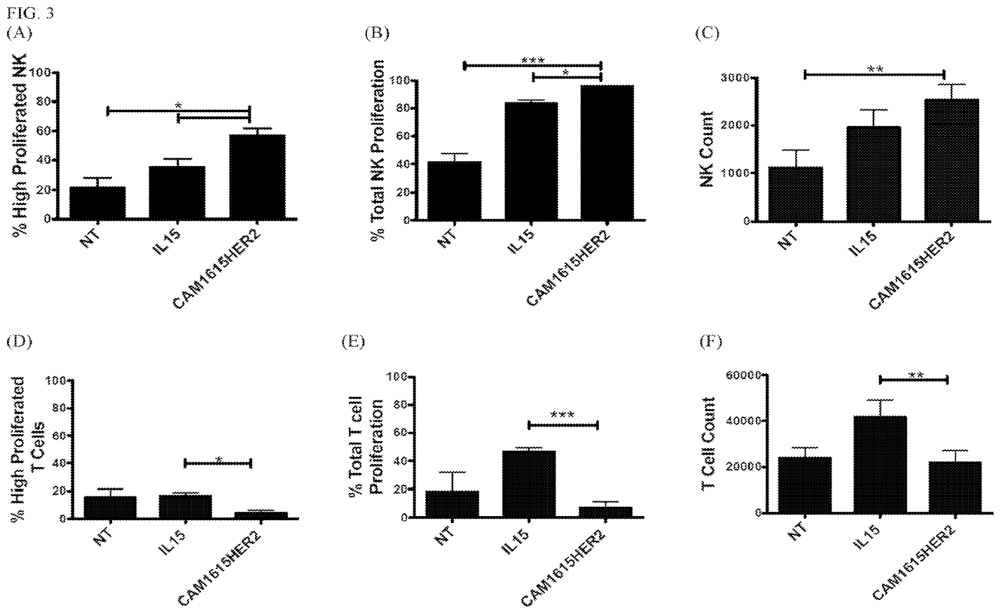

FIG. 1A shows an exemplary construction of an exemplary embodiment of second generation TriKE, referred to herein as cam1615HER2 (SEQ ID NO:1), that is capable of both antibody-dependent cellular cytotoxicity (ADCC) and NK cell expansion. The cam1615HER2 TriKE includes an anti-CD16 VHH as the NK cell engaging domain. The anti-CD16 VHH is the variable region of the heavy chain of a camelid antibody. FIG. 1B is a plasmid map showing placement of the TriKE coding sequences in a pET expression vector. FIG. 2B shows the absorbance tracing of the fractions of bacterial and target protein as it passes over FFQ ion exchange column as the first phase of purification from inclusion bodies. Eluant was collected in 8 ml aliquots. FIG. 2C shows the absorbance tracing from the second purification phase, size exclusion chromatography (SEC). The double-sided arrow shows the target peak collected as cam1615HER2 exited the column. FIG. 2A shows the final product (fractions C2-D4) as mostly a single band when analyzed using SDS-PAGE with Coomasie Blue staining providing evidence of a uniform product. The final product was greater than 90% pure with a molecular weight of about 55 kDa.

The cam1615HER2 TriKE can be constructed in other ways, however. It is possible to design multiple constructs, each of which encodes and directs synthesis of a portion of the complete immunotherapeutic compound. For example, an anti-HER2 light chain (e.g., SEQ ID NO:17) may be encoded on one plasmid and an anti-HER2 heavy chain (e.g., SEQ ID NO:18) may encoded on a second plasmid. When both plasmids are introduced into a host cell and expressed, the anti-HER2 light chain and the anti-HER2 heavy chain can dimerize to form an anti-HER2 Fab as the targeting moiety of the immunotherapeutic compound. A cam1615HER2 TriKE can be constructed in this manner. For example, SEQ ID NO:31 provides the amino acid sequence expressed from an exemplary first plasmid, the amino acid sequence containing a signal peptide, the anti-HER2 light chain, a linker, an IL-15 amino acid sequence, a second linker, and a camelid anti-CD16 single domain antibody fragment. SEQ ID NO:32 provides the amino acid sequence expressed from an exemplary second plasmid, the amino acid sequence containing a signal peptide and the anti-HER2 heavy chain.

As another example, a single plasmid construct can include all of the components of the complete immunotherapeutic compound. For example, SEQ ID NO:33 provides the amino acid sequence expressed from an exemplary single-plasmid construct, in which the amino acid sequence includes a signal sequence, the anti-HER2 heavy chain fragment, a T2A self-cleaving peptide, a second signal sequence, the anti-HER2 light chain fragment, a linker, an IL-15 amino acid sequence, a second linker, and a camelid anti-CD16 single domain antibody fragment. When expressed, the T2A peptide self-cleaves, separating the anti-HER2 heavy chain fragment from the remainder of the immunotherapeutic compound so that it is able to dimerize with the anti-HER2 light chain fragment.

In the exemplary cam1615HER2 TriKE, the human IL-15 TriKE moiety possesses the expansion capability of the molecule. Thus, the ability of the IL-15 moiety in cam1615HER2 to effect NK expansion was determined. FIG. 3A and FIG. 3B show the percentage of highly proliferated CD56⁺CD3⁻ NK cells and the total percentage of NK cells after seven days of incubation with 50 nM cam1615HER2 TriKE compared to untreated control (NT) and the IL-15 control. Both were significantly elevated after incubation with cam1615HER2. Likewise, exposure to cam1615HER2 also significantly enhanced total NK count compared to the NT control (FIG. 3C). FIG. 3D-F show that CD3⁺CD56⁻ T cell percentages and raw numbers were not elevated compared to the untreated controls. Together, these studies indicate that the HER2 TriKE stimulates expansion of NK cells but not T cells. The data also show that the IL-15 moiety within the TriKE is functional and in a viable conformational alignment.

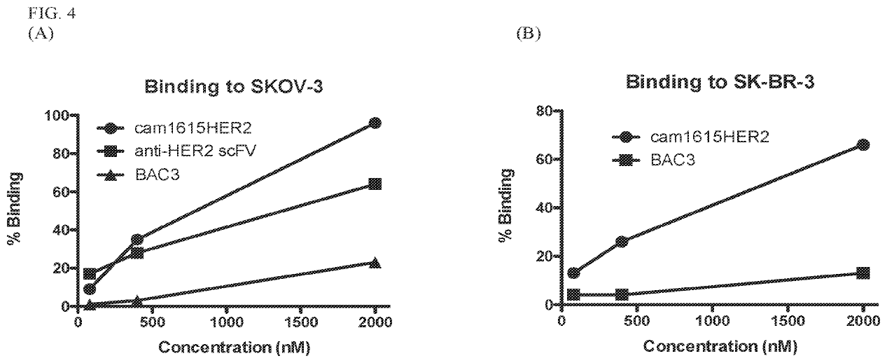

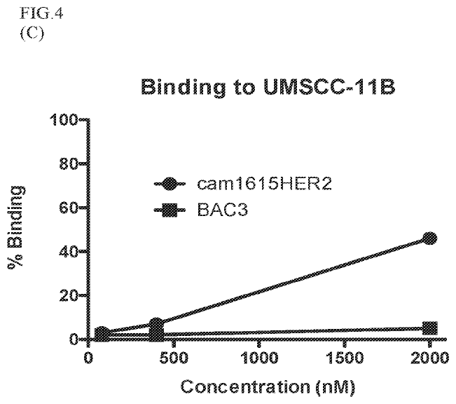

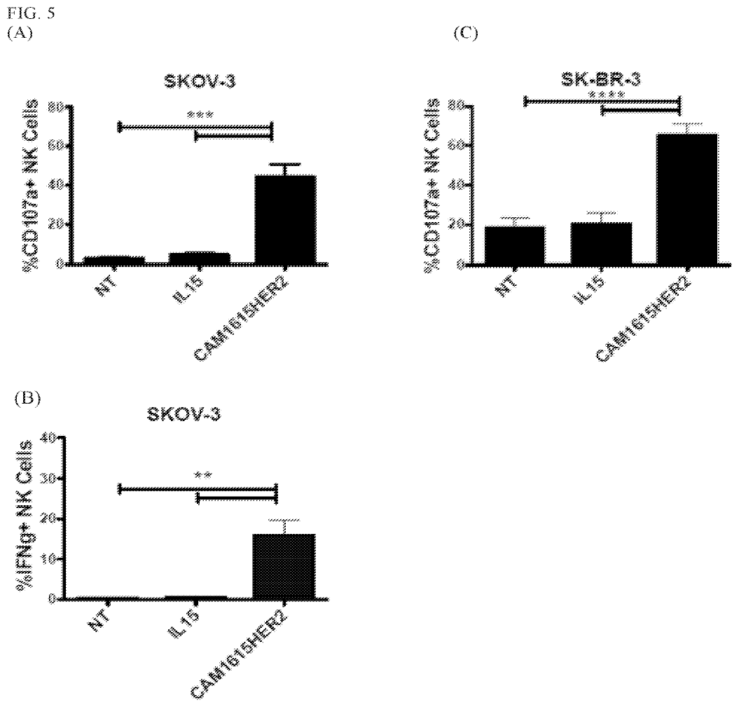

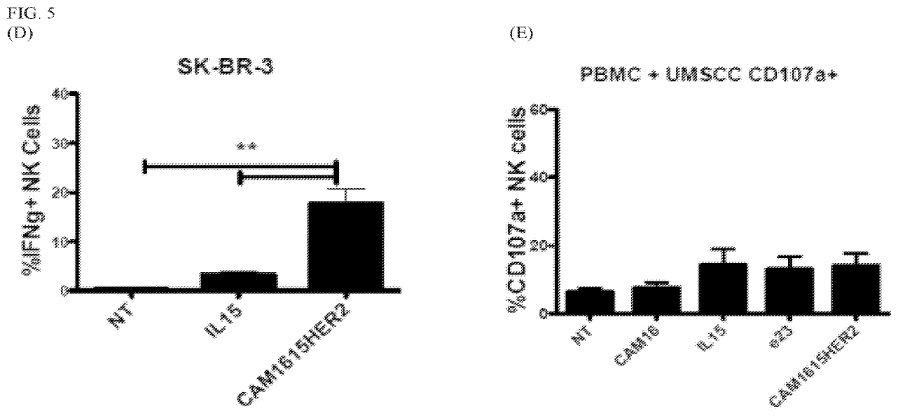

Cytotoxicity is a hallmark of NK immunotherapy. To establish the antibody-dependent cellular cytotoxicity (ADCC) potency, various cell lines were analyzed for CD107a expression, an accepted measure of NK cell cytotoxicity. The cam1615HER2 TriKE was tested against SKOV3 and SK-BR-3 since breast cancer and some ovarian cancer cases are known to overexpress ERBB2, rendering it a desirable target for antibody-directed targeting. UMSCC-11B cell line was tested as a negative control since it has minimal expression of HER2. First, binding was measured by FITC labeling the various agents and then testing their direct binding to the targets by flow cytometry. SKOV3 (FIG. 4A) and SK-BR-3 (FIG. 4B) showed the highest level of HER2 TriKE binding. UMSCC-11B showed a lower level of binding (FIG. 4C).

When ADCC activity was measured, the cam1615HER2 TriKE showed highly elevated CD107a activity against SKOV3 and SK-BR-3 cell lines compared to IL-15 and untreated controls (FIG. 5A, 5C). When ADCC was tested on the UMSCC-11B cell line, the cam1615HER2 TriKE had little effect (FIG. 5E). Also, SKOV-3 (FIG. 5B) and SK-BR-3 (FIG. 5D) cam1615HER2 showed elevated IFN-γ activation when treated with the cam1615HER2 TriKE.

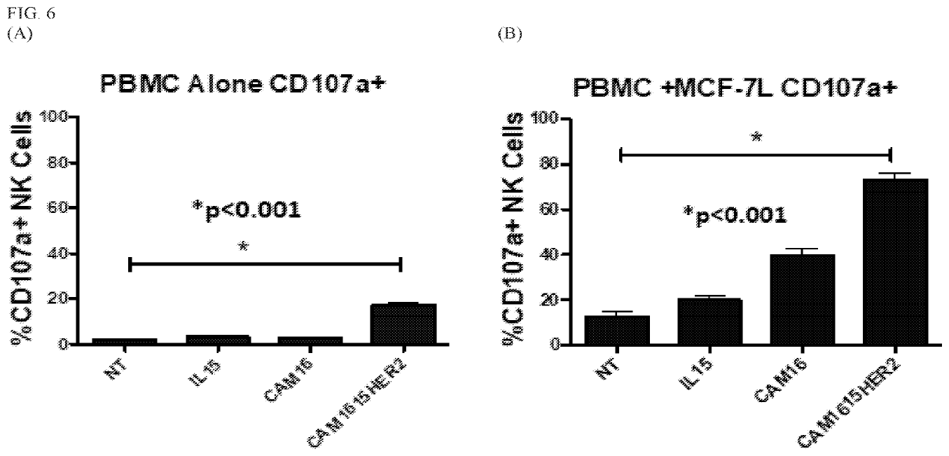

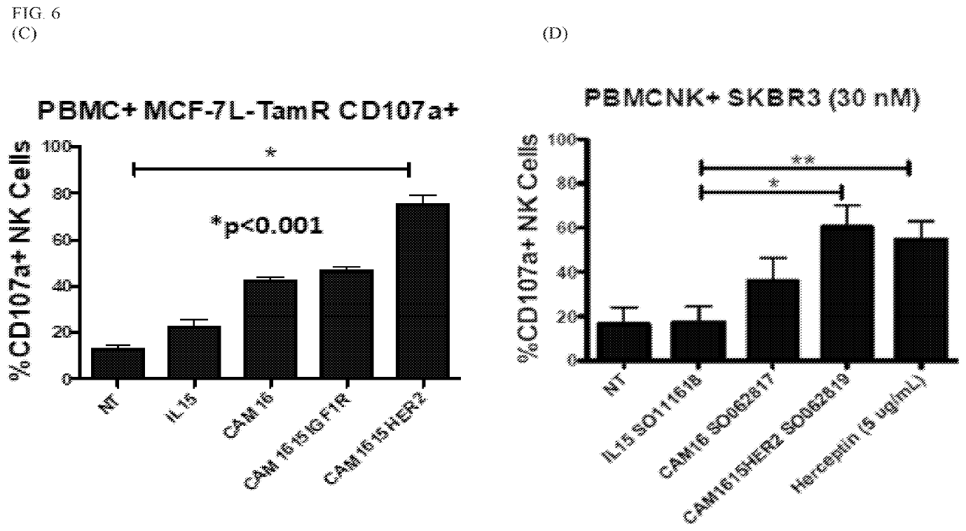

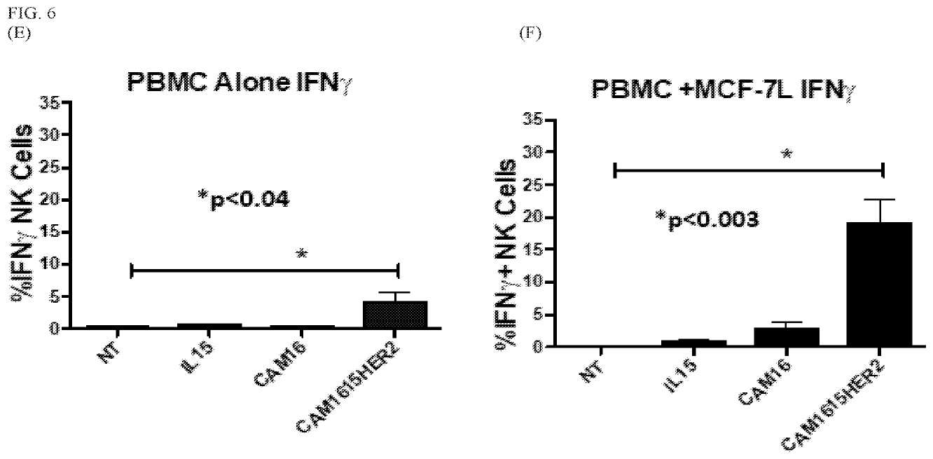

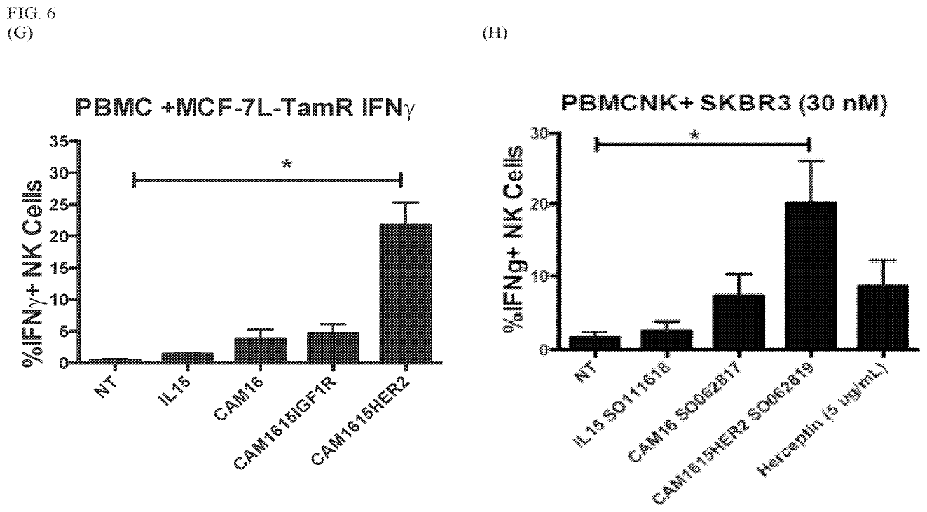

The cam1615HER2 TriKE was further tested against the breast cancer cell line MCF-7L. FIG. 6A shows CD107a effector cell background without added target cells. FIG. 6B shows CD107a activity when targets were added. The highest level of killing was observed with the cam1615HER2 TriKE compared to controls. Cam16 itself did have activity, but not as high. A subline of MCF-7L (MCF-7L-TamR) is tamoxifen resistant and showed a similar pattern of CD107a activity when treated with the cam1615HER2 TriKE (FIG. 6C). NK cells, when activated to kill, also are known for secretion of anti-cancer cytokines such as IFN-7. FIG. 6E shows IFN-γ levels, quantitated in the same samples by intracellular staining, were elevated with the cam1615HER2 TriKE and minimally affected in the controls, indicating NK cell activation. The same was true for MCF-7L-TamR (FIG. 6F). FIG. 6D confirms the cam1615HER2 TriKE killing of SK-BR-3 cells and indicates that it kills as well as trastuzumab. FIG. 6H shows that the cam1615HER2 TriKE has even greater IFN-γ enhancing activity than trastuzumab. Together, these data show that HER2 is a valid target for immune engagers on human breast cancer cells and that innate immunotherapy is highly effective in vitro against drug resistant breast cancer cell lines.

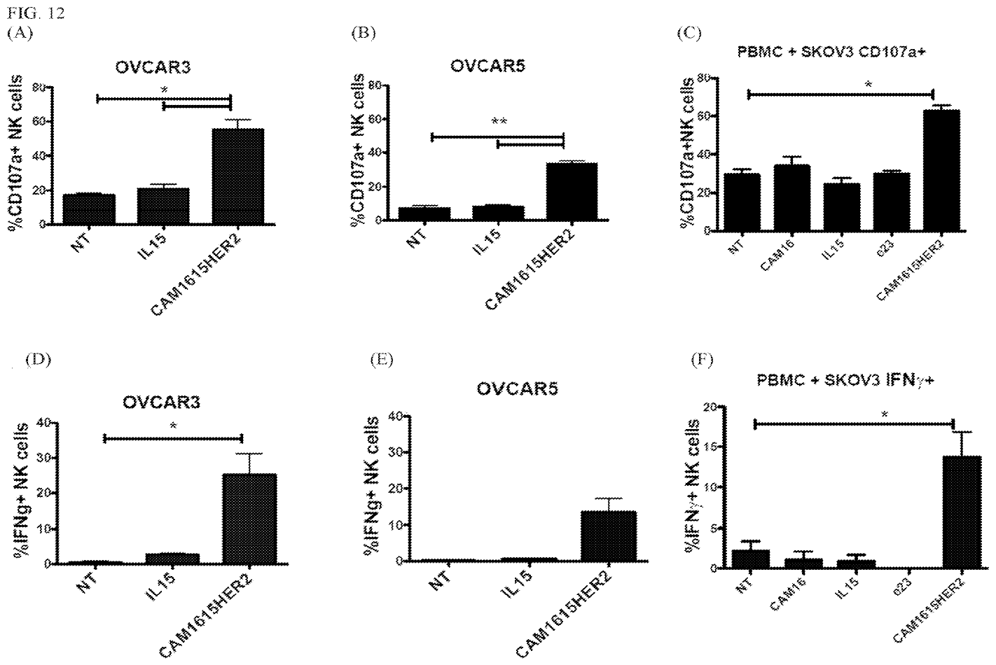

FIG. 12 provides data showing activity of cam1615HER2 TriKE against OVCAR-3 and OVCAR-5, two additional cell lines that express HER2. Again, the cam1615HER2 TriKE shows elevated CD107a activity (FIG. 12A, 12B) and IFN-γ activity (FIG. 12D, 12E) compared to controls. A repeat experiment evaluating SKOV3 is included with a more extensive number of negative controls including anti-HER2 scFv alone (e23), cam16 VHH alone, IL-15 alone, and no treatment control. Again, drug showed elevated CD107a activity (FIG. 12C) and elevated IFN-γ activity (FIG. 12F) compared to controls.

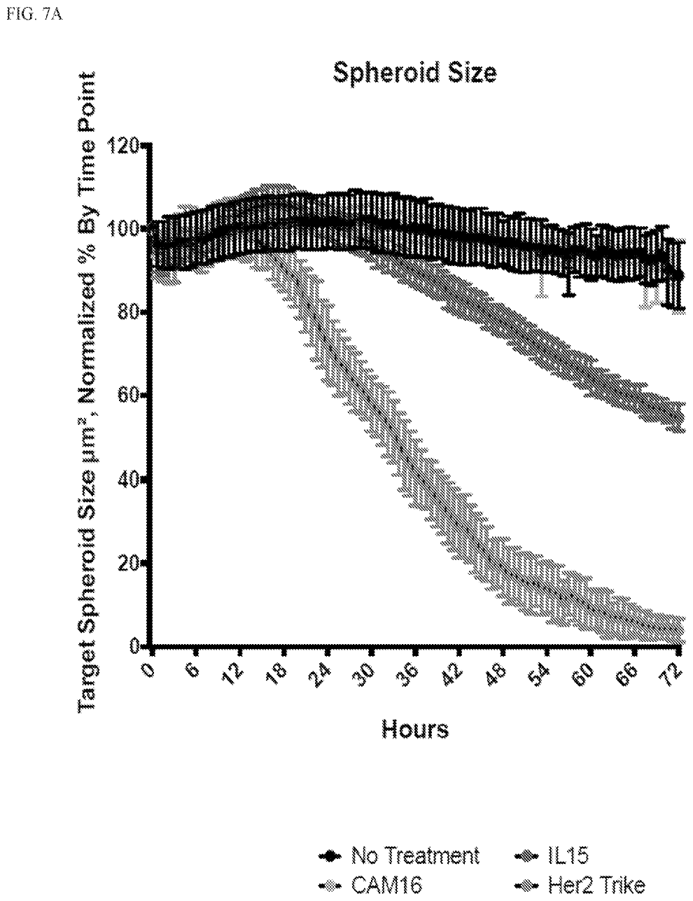

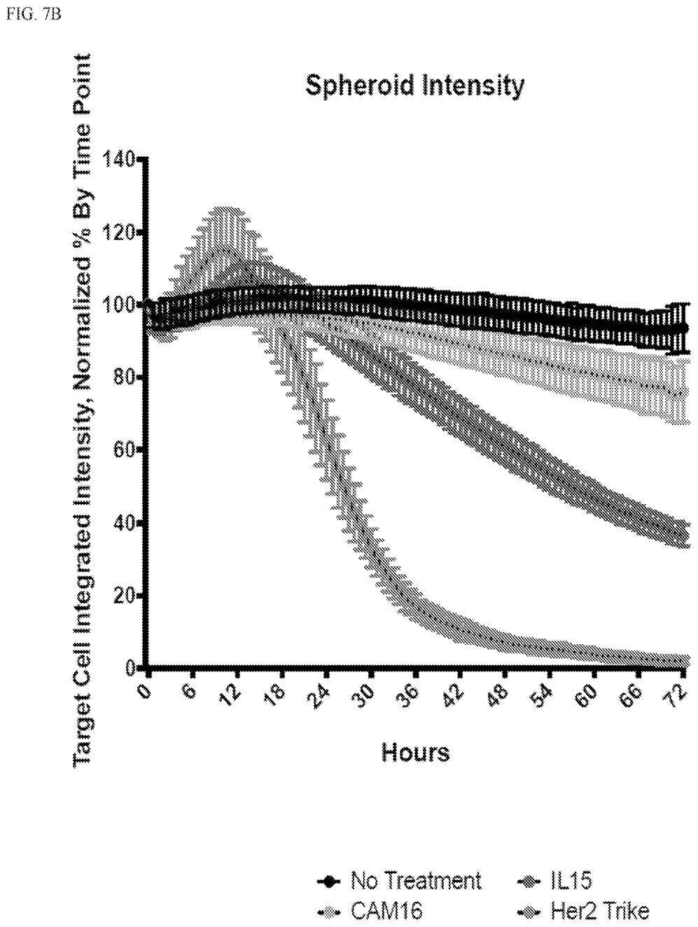

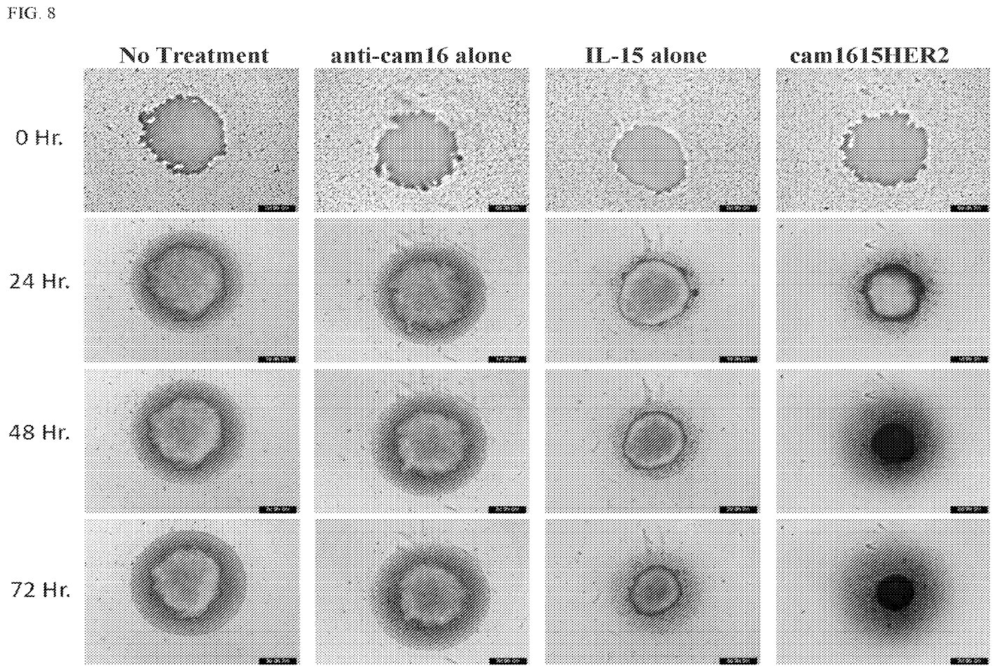

Killing was further evaluated in real time using the INCUCYTE ZOOM platform (Essen Bioscience, Inc., Ann Arbor, Mich.) over two days. SKOV3, grown in culture as spheroids were studied. SKOV3, stably transduced with NUCLIGHT RED (Essen Bioscience, Inc., Ann Arbor, Mich.) was incubated with enriched NK cells and cam1615HER2 TriKE, free IL-15, cam16 VHH alone, or no treatment. Caspase 3/7 green reagent was added to detect cell death. Dying cells become green and dying NUCLIGHT RED Raji cells become yellow, allowing for tracking of remaining living cells. When the data was compiled, cam1615HER2 TriKE induced an impressive drop in SKOV3 spheroid size (FIG. 7A) and spheroid intensity (FIG. 7B) compared to the IL-15, cam16 VHH alone, and untreated controls over the 72 hours of continuous measurement. FIG. 8 provides images showing that cam1615HER2 causes a precipitous time-dependent decline in target cells measured over a 72-hour period. Findings in this direct-kill assay correlated with findings in the CD107a assay.

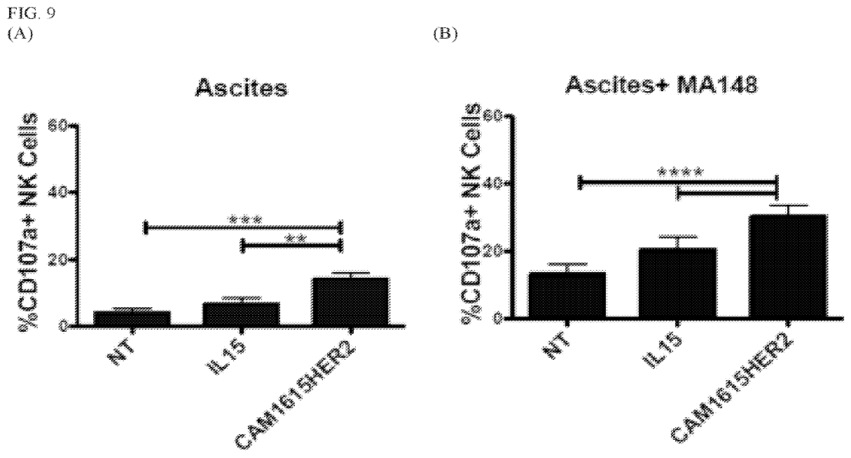

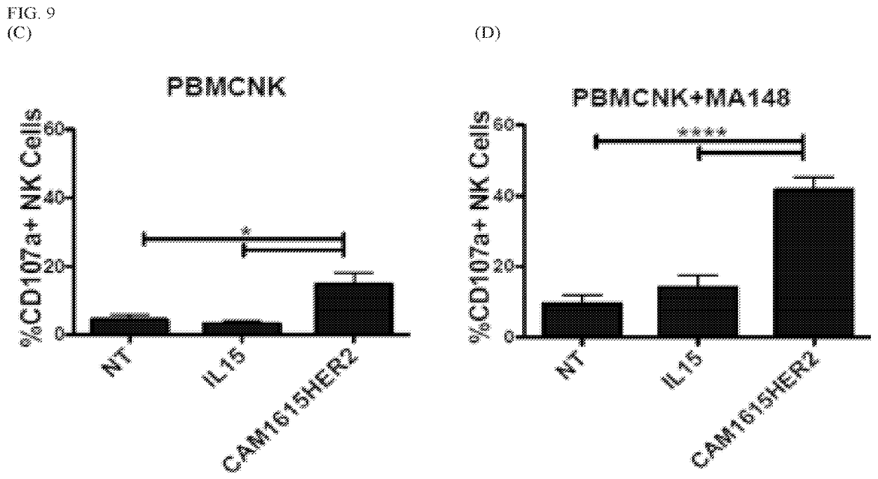

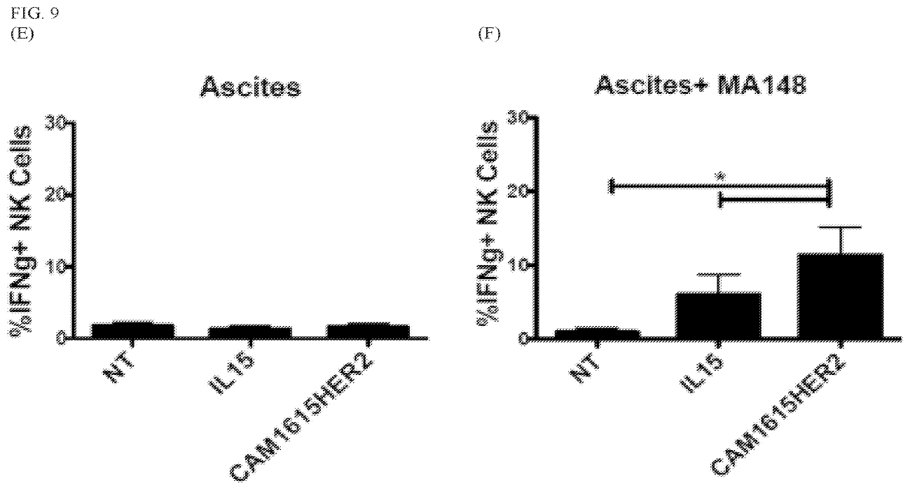

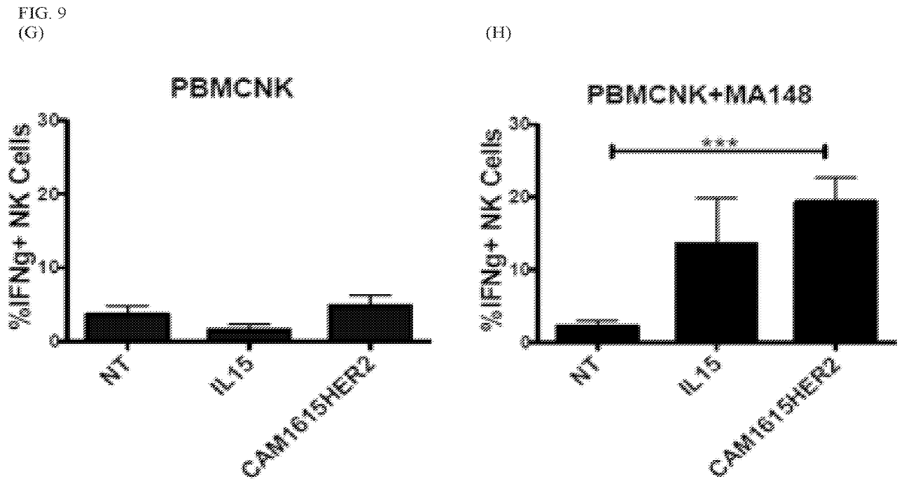

To determine whether NK cells from consented cancer patients would work in the assay, NK cells were obtained from six consented ovarian cancer patients instead of normal volunteers and tested against MA-148 ovarian cancer cells. FIG. 9A shows lower CD107a background upon treatment of effectors without cancer cells than with cam1615HER2 TriKE or controls. FIG. 9B shows significant elevation in CD107a expression in effectors plus tumor targets upon treatment with cam1615HER2 TriKE compared to untreated controls. Regarding IFN-γ activity, FIG. 9E shows low activity in effectors without targets when treated with the cam1615HER2 TriKE. When effector cells were added, the cam1615HER2 TriKE shows enhanced IFN-γ activity (FIG. 9F). For comparison, FIG. 9C shows CD107a background of normal donor effector cells and FIG. 9D shows CD107a activity of normal donor cells with MA148 cancer cells. FIG. 9G shows IFN-γ background of normal donor effector cells and FIG. 9H shows IFN-γ activity of normal donor cells with MA148 cancer cells. Although trends were similar when either normal or patient NK cells were mixed with cancer cells, the activity of normal cells was somewhat higher. Still, effector cells from patients are capable of TriKE stimulation.

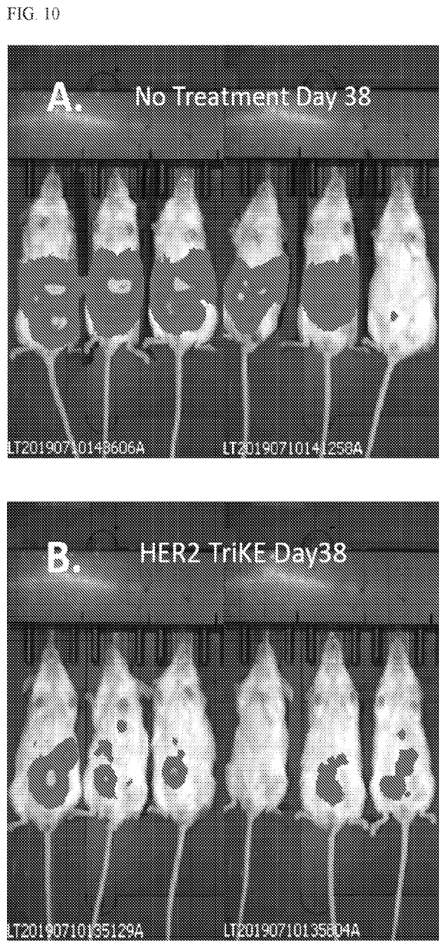

FIG. 10 shows data demonstrating in vivo efficacy of the cam1615HER2 TriKE using a SCID/hu/NK xenograft model. FIGS. 10A and 10B show visual imaging data of the untreated (FIG. 10A) and treated (FIG. 10B) groups of mice about six weeks after intraperitoneal inoculation with SKOV3 tumor cells. Advanced tumor progression was obvious in the untreated group of mice (FIG. 10A) and clearly reduced in the group of treated mice (FIG. 10B).

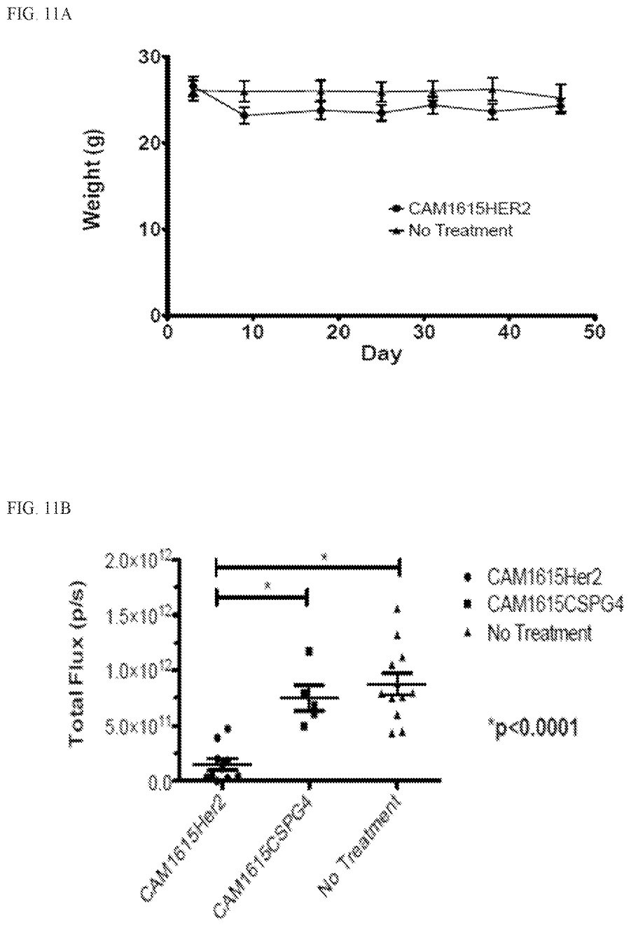

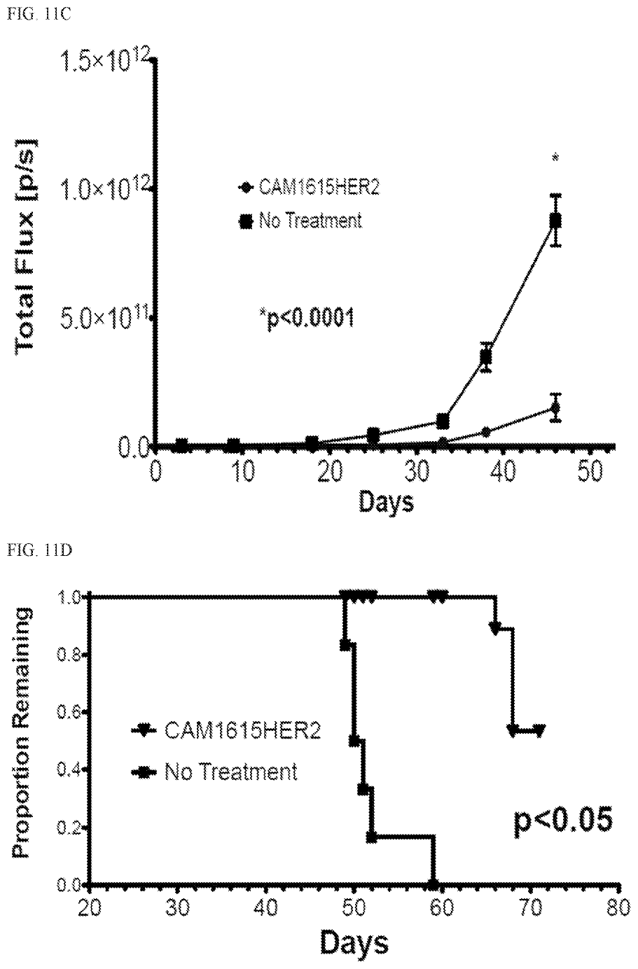

FIG. 11A shows minimal change in animal weight over a 46-day period, indicating that the cam1615HER2 TriKE is not toxic despite daily administration. FIG. 11B is a comparative snapshot of total tumor bioluminescence (total flux) from two independent experiments and shows that even on day 46 after tumor administration, tumor growth is significantly inhibited in the treated versus the untreated group. FIG. 11C shows the overall progress of tumor growth (bioluminescence) in time. Over the 46-day period, tumor growth is significantly inhibited in the treated group, but tumor growth does begin to reoccur at day 39. FIG. 11D shows longer term results in survival plot. Five of six animals in the untreated group died by day 52 and all untreated animals dies by day 60. In contrast, here is still a 50% survivorship by day 72 in the cam1615HER2-treated group. The remaining animals in the cam1615HER2-TriKE-treated group still showed tumor load, so treatment was inhibitory, not curative. Together, these data confirm that the cam1615HER2 TriKE is efficacious in inhibiting the growth of human ovarian carcinomas in vivo.

Thus, the data presented herein demonstrate that HER2 serves as an immunotherapeutic target for ovarian cancer and breast cancer when targeted by an immunotherapeutic compound such as a trispecific killer engager (TriKE) compound. An exemplary immunotherapeutic compound is effective against tamoxifen refractory cells and thus may readily kill cancer cells resistant to tamoxifen or other chemotherapeutic agents. This disclosure further demonstrates that an exemplary immunotherapeutic compound, a HER2-targeting NK engaging TriKE, can inhibit cancer in vivo in an intraperitoneal ovarian cancer xenograft model, a model that entails engrafting both human NK cells and cancer cells in xenogeneic mice. The data presented herein further provide a basis for immunotherapeutic compounds that target HER3 and/or the HER2/IER3 heterodimer.

An immunotherapeutic compound described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with an immunotherapeutic compound without causing any undesirable biological effects or interacting in a deleterious manner with any of the other components of the pharmaceutical composition in which it is contained.

An immunotherapeutic compound may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release. In certain embodiments, the composition is administered intraperitoneally, intravenously, or subcutaneously.

Thus, an immunotherapeutic compound may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like. In certain embodiments, the composition may is formulated into a solution or a suspension.

A formulation may be conveniently presented in unit dosage form and may be prepared by methods well known in the art of pharmacy. Methods of preparing a composition with a pharmaceutically acceptable carrier include the step of bringing an immunotherapeutic compound into association with a carrier that constitutes one or more accessory ingredients. In general, a formulation may be prepared by uniformly and/or intimately bringing the active molecule into association with a liquid carrier, a finely divided solid carrier, or both, and then, if necessary, shaping the product into the desired formulations.

Thus, in another aspect, this disclosure describes a method of treating cancer in a subject. Generally, the method includes administering to the subject an amount of the immunotherapeutic compound effective for treating the cancer. “Treat” or variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition; “symptom” refers to any subjective evidence of disease or of a patient's condition; and “sign” or “clinical sign” refers to an objective physical finding relating to a particular condition capable of being found by one other than the patient.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Thus, in certain embodiments, the method can involve prophylactic treatment of a subject at risk of developing a condition. “At risk” refers to a subject that may or may not actually possess the described risk. Thus, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition. Exemplary indicia of a condition can include, for example, genetic predisposition, ancestry, age, sex, geographical location, lifestyle, or medical history. Treatment may also be continued after symptoms have resolved, for example to prevent or delay their recurrence.

Accordingly, an immunotherapeutic compound may be administered to the subject before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the immunotherapeutic compound is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the immunotherapeutic compound is not administered, and/or completely resolving the condition.

The amount of the immunotherapeutic compound administered can vary depending on various factors including, but not limited to, the specific immunotherapeutic compound being administered, the weight, physical condition, and/or age of the subject, and/or the route of administration. Thus, the absolute weight of immunotherapeutic compound included in a given unit dosage form can vary widely, and depends upon factors such as the species, age, weight and physical condition of the subject, and/or the method of administration. Accordingly, it is not practical to set forth generally the amount that constitutes an amount of immunotherapeutic compound effective for all possible applications. Those of ordinary skill in the art, however, can readily determine the appropriate amount with due consideration of such factors.

In some embodiments, the method can include administering sufficient immunotherapeutic compound to provide a dose of, for example, from about 100 ng/kg/day to about 10 mg/kg/day to the subject, although in some embodiments the methods may be performed by administering an immunotherapeutic compound in a dose outside this range.

In some embodiments, the method can includes administering sufficient immunotherapeutic compound to provide a minimum dose of at least 100 ng/kg/day such as, for example, at least 1 μg/kg/day, at least 5 μg/kg/day, at least 10 μg/kg/day, at least 25 μg/kg/day, at least 50 μg/kg/day, at least 100 μg/kg/day, at least 200 μg/kg/day, at least 300 μg/kg/day, at least 400 μg/kg/day, at least 500 μg/kg/day, at least 600 μg/kg/day, at least 700 μg/kg/day, at least 800 g/kg/day, at least 900 μg/kg/day, or at least 1 mg/kg/day.

In some embodiments, the method includes administering sufficient immunotherapeutic compound to provide a maximum dose of no more than 10 mg/kg/day such as, for example, no more than 5 mg/kg/day, no more than 4 mg/kg/day, no more than 3 mg/kg/day, no more than 2 mg/kg/day, no more than 1 mg/kg/day, no more than 900 μg/kg/day, no more than 800 g/kg/day, no more than 700 μg/kg/day, no more than 600 μg/kg/day, no more than 500 ag/kg/day, no more than 400 μg/kg/day, no more than 300 μg/kg/day, no more than 200 g/kg/day, no more than 100 μg/kg/day, no more than 90 μg/kg/day, no more than 80 μg/kg/day, no more than 70 μg/kg/day, no more than 60 μg/kg/day, no more than 50 μg/kg/day, no more than 40 μg/kg/day, no more than 30 μg/kg/day, no more than 20 μg/kg/day, or no more than 10 g/kg/day. The immunotherapeutic compound provides a dose of “no greater than” a specified amount when the immunotherapeutic compound is not absent but is present in an amount up to and including the specified amount.

In some embodiments, the method includes administering sufficient immunotherapeutic compound to provide a dose characterized by a range having endpoints defined by any a minimum dose identified above and any maximum dose that is greater than the selected minimum dose. For example, in some embodiments, the method can include administering sufficient immunotherapeutic compound to provide a dose of from about 10 μg/kg/day to about 10 mg/kg/day to the subject, a dose of from about 100 μg/kg/day to about 1 mg/kg/day, a dose of from 5 μg/kg/day to 100 μg/kg/day, etc.

In certain embodiments, the method includes administering sufficient immunotherapeutic compound to provide a dose that is equal to any minimum dose or any maximum dose listed above. Thus, for example, in certain embodiments, the method can include administering sufficient immunotherapeutic compound to provide a dose of 1 μg/kg/day, 5 μg/kg/day, 10 μg/kg/day, 25 μg/kg/day, 50 μg/kg/day, 100 μg/kg/day, 200 μg/kg/day, 500 μg/kg/day, 1 mg/kg/day, 5 mg/kg/day, etc.

In some embodiments, an immunotherapeutic compound may be administered, for example, from a single dose to multiple doses per week, although in some embodiments the method can be performed by administering an immunotherapeutic compound at a frequency outside this range. In certain embodiments, an immunotherapeutic compound may be administered from about once per month to about five times per week. In some embodiments, the doses indicated above, which are described in terms of the amount of immunotherapeutic compound administered over a 24-hour period, are administered in a seven-day cycle of four days of treatment and three days of rest.

In some embodiments, an immunotherapeutic compound may be administered, for example, from a single dose to multiple cycles of treatment, although in some embodiments the method can be performed by administering an immunotherapeutic compound for a duration outside this range. In some embodiments, the immunotherapeutic compound may be administered for three weeks. In such embodiments, each week may be a treatment cycle such as the exemplary treatment cycle described in the preceding paragraph. In other embodiments, the immunotherapeutic compound may be administered for a greater number of treatment cycles, without a gap between one set of treatment cycles and a subsequent set of treatment cycles. The gap between one set of treatment cycles and a subsequent set of treatment cycles may be a gap of one or more weeks, one or more months, or one or more years.

In some embodiments, the method further includes administering one or more additional therapeutic agents. The one or more additional therapeutic agents (e.g., chemotherapeutic agents) may be administered before, after, and/or coincident to the administration of an immunotherapeutic compound. An immunotherapeutic compound and the additional therapeutic agents may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. In other embodiments, the immunotherapeutic compound and the additional therapeutic agent may be administered as part of a mixture or cocktail. In some aspects, the administration of the immunotherapeutic compound may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents include altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, and vinorelbine, an anti-HER2 antibody therapy, an anti-HER3 antibody therapy (see, e.g., Liu et al., 2019, Biol Proced Online 21:5), or an anti-HER2/IER3 heterodimer complex antibody therapy (see, e.g., Liu et al., 2019, Biol Proced Online 21:5).

In some embodiments, of the method can include administering sufficient immunotherapeutic compound as described herein and administering the at least one additional therapeutic agent demonstrate therapeutic synergy. In some aspects of the methods of the present invention, a measurement of response to treatment observed after administering both an immunotherapeutic compound as described herein and the additional therapeutic agent is improved over the same measurement of response to treatment observed after administering either the immunotherapeutic compound or the additional therapeutic agent alone. In some embodiments, an additional therapeutic agent can include an additional agent that targets EpCAM including, for example, an EpCAM specific monoclonal antibody, such as, for example, catumaxomab, a monoclonal hybrid antibody targeting EpCAM and CD3.

In some embodiments, administering the immunotherapeutic compound to a subject can stimulate endogenous NK cells in vivo. Using an immunotherapeutic compound as a part of an in vivo method can make NK cells antigen specific with simultaneous co-stimulation, enhancement of survival, and expansion. In other cases, the immunotherapeutic compound can be used in vitro as an adjuvant to NK cell adoptive transfer therapy.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Examples

Construction of cam11615HER TriKEs

DNA fragments encoding the CDR regions from a camelized anti-CD16 (Vincke et al., 2009, J. Biol. Chem. 284(5):3273-3284) were spliced into a universal, humanized nanobody scaffold previously shown to allow grafts of antigen-binding loops with transfer of the antigen specificity and affinity (Behar et al., 2008, Protein Ehgineering, Design & Selection 21(1):1-10). This new sequence was used to manufacture cam1615HER2 (SEQ ID NO: 1). The fully assembled hybrid gene cam11615THR2, which encodes the cam 1615HER2 TriKE, encodes (from 5′ end to 3′ end) a NcoI restriction site; an ATG start codon; anti-human CD16 VHH; a 20-amino-acid (aa) segment, PSGQAGAAASESLFVSNHAY (SEQ ID NO:3); human IL-15; the seven amino acid linker, EASGGPE (SEQ ID NO:5); anti-HER2 scFv (Batra et al., 1992, Proc Natl Acad Sci USA 89(13):5867-5871), and a XhoI restriction site. The resulting hybrid gene (SEQ ID NO:7) was spliced into the pET28c expression vector under the control of an isopropyl-D-thiogalactopyranoside (IPTG) inducible 17 promoter. The DNA target gene encoding cam1615IER2 was 1517 base pairs. Wild-type human IL-15 was used and not a mutated form of the cytokine. The Biomedical Genomics Center, University of Minnesota, St. Paul, Minn. verified the gene sequence and in-frame accuracy of the target gene.

Purification of Protein from Inclusion Bodies

Escherichia coli strain. BL21 (DE3) (Novagen, Madison, Wis.) was used for protein expression after plasmid transfection. The bacteria were cultured overnight in 800-ml Luria broth containing 50 μg/ml kanamycin. Expression was induced by adding IPTG (Thermo Fisher Scientific, Inc., Fair Lawn, N.J.) when the media reached an absorbance of 0.65 at 600 nm. Bacterial expression resulted in packaging of target protein into inclusion bodies. After expression, bacteria were harvested and then homogenized in buffer (50 mM Tris, 50 mM NaCl, and 5 mM EDTA pH 8.0) and the pellet sonicated and centrifuged. To extract protein from the pellet a solution of 0.3% sodium deoxycholate, 5% Triton X-100, 10% glycerin, 50 mmol/L Tris, 50 mmol/L NaCl, and 5 mmol/L EDTA (pH 8.0), was used and the extract was washed three times.

Protein from inclusion bodies requires refolding. Thus, a sodium N-lauroyl-sarcosine (SLS) air oxidation method was used that is modified from a previously described method (Vallera et al., 2005, Leuk Res 29(3):331-341). Briefly, inclusion bodies were dissolved in 100 mM Tris, 2.5% SLS (Sigma-Aldrich, St. Louis, Mo.). Pellets were removed by centrifugation. 50 M of CuSO₄ was added to the solution and then incubated at room temperature with rapid stirring for 20 hours for air-oxidization of —SH groups. Removal of SLS was performed by adding 6 M urea and 10% AG 1-X8 resin (200-400 mesh, chloride form) (Bio-Rad Laboratories, Inc., Hercules, Calif.) to the detergent-solubilized protein solution. The next step added 13.3 M guanidine-HCl into the protein solution, followed by incubation at 37° C. for two to three hours. The solution was diluted 20-fold with refolding buffer (50 mM Tris, 0.5 M L-arginine, 1 M urea, 20% glycerol, 5 mM EDTA, pH 8.0). The mixture was incubated at 4° C. for two days. To remove the buffer, the sample was dialyzed against five volumes of 20 mM Tris-HCl at pH 8.0 for 48 hours at 4° C., then eight volumes for 18 additional hours. The product was then purified first by fast flow Q ion exchange chromatography and then by passage over a size exclusion column (SUPERDEX 200, Cytiva, Marlborough, Mass.). Sodium dodecyl sulfate polyacrylamide gel electrophoresis (SDS-PAGE) was performed using Simply Blue life Stain (Invitrogen, Carlsbad, Calif.) to evaluate protein size and purity.

### Cancer Cell Lines

The following cell lines were obtained from the American Type Culture Collection (Manassas, Va.): MCF-7L (ductal breast carcinoma), MCF-7L-TamR (tamoxifen-resistant subline of MCF-7), SKOV3 (ovarian ascites), SK-BR-3 (breast carcinoma derived from a metastatic site), UMSCC-11B squamous cell carcinoma derived from a larynx tumor (Worsham et al., 2006, Arch Otolaryngol Head Neck Surg 132:668-677), HL-60 (acute promyelocytic leukemia), MA-148 (ovarian carcinoma), and SKOV3-luc. For in vivo experiments, SKOV3-luc was made by transfecting SKOV3 with a luciferase reporter construct using LIPOFECTAMINE (Invitrogen, Carlsbad, Calif.) and selective pressure applied with 10 μg/ml of blastocidin. UMSCC-11B was authenticated by STR testing performed by the Fragment Analysis Facility, John Hopkins University. MA148 (established locally at the University of Minnesota) is a human epithelial ovarian carcinoma cell line. Lines were maintained in RPMI 1640 RPMI supplemented with 10-20% fetal bovine serum (FBS) and 2 mmol/L L-glutamine. Lines were incubated in a humidified atmosphere containing 5% CO₂ at a constant 37° C. When the adherent cells were more than 90% confluent, they were passaged using trypsin-EDTA for detachment. For the cell counts a standard hemocytometer was used. Only those cells with a viability >95% were used for the experiments, as determined by trypan blue exclusion.

### Evaluation of Cytotoxicity and NK Cell Activation

Antibody-dependent cellular cytotoxicity (ADCC) was measured using a CD107a (lysosomal-associated membrane protein LAMP-1) flow cytometry assay. For effector cells, PBMC were obtained from normal volunteers after procuring donor consent and permissions of the Institutional Review Board. Cancer target cells were grown from cell lines as described above. For experiments using patient effector cells, the University of Minnesota Cancer Center Tissue Procurement Facility obtained high-grade serous ascites samples from patients diagnosed with ovarian cancer following approval from the Institutional Review Board. All specimens were collected from women diagnosed with advanced-stage ovarian or primary peritoneal carcinoma at time of primary debulking surgery. Cells were pelleted, lysed to remove red blood cells, cryopreserved in 10% DMSO/90% FBS and stored in liquid nitrogen.

PBMCs were incubated overnight (37° C., 5% C02) in RPMI 1640 media supplemented with 10% fetal calf serum (RPMI-10) and suspended with tumor target cells or media after thrice washing with RPMI-10. Cells were then incubated with TriKEs or controls for 10 minutes at 37° C. Fluorescein isothiocyate (FITC)-conjugated anti-human CD107a monoclonal antibody (BD Biosciences, San Jose, Calif.) was then added and incubated for one hour. After incubation, GolgiStop (1:1,500, BD Biosciences, San Jose, Calif.) and GolgiPlug (1:1,000, BD Biosciences, San Jose, Calif.) were added for three hours (37° C., 5% C02). After the washing with phosphate buffered saline, the cells were stained with PE/Cy7-conjugated anti-CD56 mAb, APC/Cy7-conjugated anti-CD16 mAb, and PE-CF594-conjugated anti-CD3 mAb (BioLegend, San Diego, Calif.). Cells were incubated for 15 minutes at 4° C., washed, and fixed with 2% paraformaldehyde.

Intracellular IFN-γ was measured as an indicator of NK cell activation. Briefly, cells were exposed to permeabilization buffer (BD Biosciences, San Jose, Calif.) and incubated with Pacific Blue-conjugated antihuman IFN-γ (BioLegend, San Diego, Calif.) for 20 minutes. The cells were finally washed and evaluated by fluorescence-activated cell sorting analysis using a LSRII flow cytometer (BD Biosciences, San Jose, Calif.) gating on CD56⁺CD3⁻ cells.

Cytotoxicity potency was additionally measured in real time. Magnetic bead enriched CD56⁺CD3⁻ NK effector cells were plated into 96-well flat clear bottom polystyrene tissue-culture treated microplates (Corning, Flintshire, UK) along with red labeled tumor cells and the plates were transferred into the INCUCYTE ZOOM platform (Essen Bioscience, Inc., Ann Arbor, Mich.) which was housed inside a cell incubator at 37° C./5% C02. Images from three technical replicates were taken every 15 minutes for 48 hours using a 4× objective lens and then analyzed using the INCUCYTE Basic Software (Essen Bioscience, Inc., Ann Arbor, Mich.).

### NK Cell Expansion Via IL-15 Stimulation

To measure TriKE potency that depends on its functional IL-15 moiety, PBMCs or enriched NK cells from healthy donors were labeled with CELLTRACE violet proliferation dye (Invitrogen, Carlsbad, Calif.) according to kit specifications. After staining, effector cells were cultured with 50 nM of TriKEs or controls and incubated in a humidified atmosphere containing 5% C02 at 37° C. for seven days. Cells were harvested, stained for viability with Live/Dead reagent (Invitrogen, Carlsbad, Calif., USA) and surface stained for anti-CD56 PE/Cy7 (Biolegend, SanDiego, Calif.) and anti-CD3 PE-CF594 (BD Biosciences, Franklin Lakes, N.J.) to gate on the viable CD3-CD56+NK cell population. Data analysis was performed using FlowJo software (Flowjo enterprise LCC, version 7.6.5, Ashland, Oreg.).

### In Vivo Mouse Study and Imaging

The efficacy of the HER2 TriKE was tested in a scid/hu mouse model that was previously reported (Vallera et al., 2016, Clin Cancer Res 22(14):3440-3450), but modified for the growth of the human ovarian cancer cell line SCOV3. The line was transfected with a luciferase reporter gene to permit monitoring of tumor progression via bioluminescent imaging in real time. NSG mice (NOD.Cg-Prkdcs^{cid} Il2rg^{tm1Wwjl}/SzJ, n=5/group) were injected intraperitoneally with 2×10⁵ SCOV3 cells and then 3-5 days later received low-dose total body irradiation (275 cGy). The following day, all groups received highly enriched NK cells (PBMC magnetically CD3 and CD19 depleted) and began treatment with the cam1615HER2 TriKE. A single course of treatment consisted of 50 μg of drug given intraperitoneally five times per week (MTWThF) for two weeks and then maintenance therapy three times per week (MWF) through day 60. Live mice were imaged weekly. At each imaging session, mice were injected with 100 l of 30 mg/ml luciferin substrate for 10 minutes and then imaged under isoflurane gas sedation. Imaging data were gathered using Xenogen Ivis 100 imaging system with Living Image 2.5 software (Xenogen Corporation, Hopkington Mass.). Mice were weighed weekly when possible.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1 - cam1615HER2 Amino Acid Sequence:
MEQVQLVESG GGLVQPGGSL RLSCAASGLT FSSYNMGWFR QAPGQGLEAV ASITWSGRDT
FYADSVKGRF TISRDNSKNT LYLQMNSLRA EDTAVYYCAA NPWPVAAPRS GTYWGQGTLV
TVSSPSGQAG AAASESLFVS NHAYNWVNVI SDLKKIEDLI QSMHIDATLY TESDVHPSCK
VTAMKCFLLE LQVISLESGD ASIHDTVENL IILANNSLSS NGNVTESGCK ECEELEEKNI
KEFLQSFVHI VQMFINTSEA SGGPEDVQLT QSPAILSASP GEKVTMTCRA TPSVSYMHWY
QQKPGSSPKP WIYTTSNLAS GVPARFSGGG SGTSYSLTVS RVEAEDAATY YCQQWSRSPP
TFGGGSKLEI KGSTSGSGKS SEGKGVQLQE SGPEVVKPGG SMKISCKTSG YSFTGHTMNW
VKQSHGKNLE WIGLINPYNG DTNYNQKFKG KATFTVDKSS STAYMELLSL TSEDSAVYYC
ARRVTDWYFD VWGAGTTVTV S
SEQ ID NO 2 - cam16 (amino acids 3-124 of SEQ ID NO: 1)
QVQLVESGGG LVQPGGSLRL SCAASGLTFS SYNMGWFRQA PGQGLEAVAS ITWSGRDTFY
ADSVKGRFTI SRDNSKNTLY LQMNSLRAED TAVYYCAANP WPVAAPRSGT YWGQGTLVTV
SS
SEQ ID NO: 3 - hma linker (amino acids 125-144 of SEQ ID NO: 1)
PSGQAGAAAS ESLFVSNHAY
SEQ ID NO: 4- human IL-15 (amino acids 145-258 of SEQ ID NO: 1)
NWVNVISDLK KIEDLIQSMH IDATLYTESD VHPSCKVTAM KCFLLELQVI SLESGDASIH
DTVENLIILA NNSLSSNGNV TESGCKECEE LEEKNIKEFL QSFVHIVQMF INTS
SEQ ID NO: 5 - linker (amino acids 259-265 of SEQ ID NO: 1)
EASGGPE
SEQ ID NO: 6 - anti-HER2 (amino acids 266-501 of SEQ ID NO: 1)
DVQLTQSPAI LSASPGEKVT MTCRATPSVS YMHWYQQKPG SSPKPWIYTT SNLASGVPAR
FSGGGSGTSY SLTVSRVEAE DAATYYCQQW SRSPPTFGGG SKLEIKGSTS GSGKSSEGKG
VQLQESGPEV VKPGGSMKIS CKTSGYSFTG HTMNWVKQSH GKNLEWIGLI NPYNGDTNYN
QKFKGKATFT VDKSSSTAYM ELLSLTSEDS AVYYCARRVT DWYFDVWGAG TTVTVS
SEQ ID NO: 7 - cam1615HER2 DNA sequence
CCATGGAGca ggtgcagctg gtggagtctg ggggaggctt ggtgcagcct gggggctctc
tgagactctc ctgtgcagcc tctggcctca ccttcagtag ctataacatg ggctggttcc
gccaggctcc agggcaaggc cttgaggctg tagcatctat tacctggagt ggtcgggaca
cattctatgc agactccgtg aagggccgat tcaccatctc cagagacaac tccaagaaca
ctctctatct gcaaatgaac agcctgcgcg cggaggacac ggccgtttat tattgtgctg
caaacccctg gccagtggcg gcgccacgta gtggcaccta ctggggccaa gggaccctgg
tcaccgtctc ctcaccgtct ggtcaggctg gtgctgctgc tagcgaatct ctgttcgttt
ctaaccacgc ttacAACTGG GTGAATGTAA TAAGTGATTT GAAAAAAATT GAAGATCTTA
TTCAATCTAT GCATATTGAT GCTACTTTAT ATACGGAAAG TGATGTTCAC CCCAGTTGCA
AAGTAACAGC AATGAAGTGC TTTCTCTTGG AGTTACAAGT TATTTCACTT GAGTCCGGAG
ATGCAAGTAT TCATGATACA GTAGAAAATC TGATCATCCT AGCAAACAAC AGTTTGTCTT
CTAATGGGAA TGTAACAGAA TCTGGATGCA AAGAATGTGA GGAACTGGAG GAAAAAAATA
TTAAAGAATT TTTGCAGAGT TTTGTACATA TTGTCCAAAT GTTCATCAAC ACTTCTgaag
cttccggagg tcccgagGAC GTCCAGCTGA CCCAGTCTCC AGCAATCCTG TCTGCATCTC
CAGGGGAGAA GGTCACAATG ACTTGCAGGG CCACCCCAAG TGTAAGTTAC ATGCACTGGT
ATCAGCAGAA GCCAGGATCC TCCCCCAAAC CTTGGATTTA TACCACATCC AACCTGGCTT
CTGGAGTCCC TGCTCGCTTC AGTGGCGGTG GGTCTGGGAC CTCTTACTCT CTCACAGTCA
GCAGAGTGGA GGCTGAAGAT GCTGCCACTT ATTACTGCCA GCAGTGGAGT CGTAGCCCAC
CCACGTTCGG AGGGGGGTCC AAGCTGGAAA TAAAAGGTTC TACCTCTGGT TCTGGTAAAT
CTTCTGAAGG TAAAGGTGTG CAGCTGCAGG AGTCAGGACC TGAGGTGGTG AAGCCTGGAG
GTTCAATGAA GATATCCTGC AAGACTTCTG GTTACTCATT CACTGGCCAC ACCATGAACT
GGGTGAAGCA GAGCCATGGA AAGAACCTTG AGTGGATTGG ACTTATTAAT CCTTACAATG
GTGATACTAA CTACAACCAG AAGTTCAAGG GCAAGGCCAC ATTTACTGTA GACAAGTCGT
CCAGCACAGC CTACATGGAG CTCCTCAGTC TGACATCTGA GGACTCTGCA GTCTATTACT
GTGCAAGGAG GGTTACGGAC TGGTACTTCG ATGTCTGGGG CGCAGGGACC ACGGTCACCG
TCTCCtaata gctcgag
SEQ ID NO: 8 - NcoI restriction site and start codon (1-8 of SEQ ID NO: 7)
CCATGGAG
SEQ ID NO: 9 - cam16 (9-374 of SEQ ID NO: 7)
caggtgcagc tggtggagtc tgggggaggc ttggtgcagc ctgggggctc tctgagactc
tcctgtgcag cctctggcct caccttcagt agctataaca tgggctggtt ccgccaggct
ccagggcaag gccttgaggc tgtagcatct attacctgga gtggtcggga cacattctat
gcagactccg tgaagggccg attcaccatc tccagagaca actccaagaa cactctctat
ctgcaaatga acagcctgcg cgcggaggac acggccgttt attattgtgc tgcaaacccc
tggccagtgg cggcgccacg tagtggcacc tactggggcc aagggaccct ggtcaccgtc
tcctca
SEQ ID NO: 10 - hma linker (375-434 of SEQ ID NO: 7)
ccgtctggtc aggctggtgc tgctgctagc gaatctctgt tcgtttctaa ccacgcttac
SEQ ID NO: 11 - human IL-15 (435-776 of SEQ ID NO: 7)
AACTGGGTGA ATGTAATAAG TGATTTGAAA AAAATTGAAG ATCTTATTCA ATCTATGCAT
ATTGATGCTA CTTTATATAC GGAAAGTGAT GTTCACCCCA GTTGCAAAGT AACAGCAATG
AAGTGCTTTC TCTTGGAGTT ACAAGTTATT TCACTTGAGT CCGGAGATGC AAGTATTCAT
GATACAGTAG AAAATCTGAT CATCCTAGCA AACAACAGTT TGTCTTCTAA TGGGAATGTA
ACAGAATCTG GATGCAAAGA ATGTGAGGAA CTGGAGGAAA AAAATATTAA AGAATTTTTG
CAGAGTTTTG TACATATTGT CCAAATGTTC ATCAACACTT CT
SEQ ID NO: 12 - Linker (777-797 of SEQ ID NO: 7)
gaagcttccg gaggtcccga g
SEQ ID NO: 13 - anti-HER2 (798-1505 of SEQ ID NO: 7)
GACGTCCAGC TGACCCAGTC TCCAGCAATC CTGTCTGCAT CTCCAGGGGA GAAGGTCACA
ATGACTTGCA GGGCCACCCC AAGTGTAAGT TACATGCACT GGTATCAGCA GAAGCCAGGA
TCCTCCCCCA AACCTTGGAT TTATACCACA TCCAAGCTGG CTTCTGGAGT CCCTGCTCGC
TTCAGTGGCG GTGGGTCTGG GACCTCTTAC TCTCTCACAG TCAGCAGAGT GGAGGCTGAA
GATGCTGCCA CTTATTACTG CCAGCAGTGG AGTCGTAGCC CACCCACGTT CGGAGGGGGG
TCCAAGCTGG AAATAAAAGG TTCTACCTCT GGTTCTGGTA AATCTTCTGA AGGTAAAGGT
GTGCAGCTGC AGGAGTCAGG ACCTGAGGTG GTGAAGCCTG GAGGTTCAAT GAAGATATCC
TGCAAGACTT CTGGTTACTC ATTCACTGGC CACACCATGA ACTGGGTGAA GCAGAGCCAT
GGAAAGAACC TTGAGTGGAT TGGACTTATT AATCCTTACA ATGGTGATAC TAACTACAAC
CAGAAGTTCA AGGGCAAGGC CACATTTACT GTAGACAAGT CGTCCAGCAC AGCCTACATG
GAGCTCCTCA GTCTGACATC TGAGGACTCT GCAGTCTATT ACTGTGCAAG GAGGGTTACG
GACTGGTACT TCGATGTCTG GGGCGCAGGG ACCACGGTCA CCGTCTCC
SEQ ID NO: 14 - Two stop codons and XhoI restriction site (1506-1517 of SEQ ID NO: 7)
taatagctcg aga
SEQ ID NO: 15 - e23 anti-HER2 amino acid sequence
DVQLTQSPAI LSASPGEKVT MTCRATPSVS YMHWYQQKPG SSPKPWIYTT SNLASGVPAR
FSGGGSGTSY SLTVSRVEAE DAATYYCQQW SRSPPTFGGG SKLEIKGSTS GSGKSSEGKG
VQLQESGPEV VKPGGSMKIS CKTSGYSFTG HTMNWVKQSH GKNLEWIGLI NPYNGDTNYN
QKFKGKATFT VDKSSSTAYM ELLSLTSEDS AVYYCARRVT DWYFDVWGAG TTVTVS
SEQ ID NO: 16 - Trastuzumab-based scFv
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
GSTSGSGKPG SGEGSTKGDI QMTQSPSSLS ASVGDRVTIT CRASQDVNTA VAWYQQKPGK
APKLLIYSAS FLYSGVPSRF SGSRSGTDFT LTISSLQPED FATYYCQQHY TTPPTFGQGT
KVEIKRTV
SEQ ID NO: 17 - Anti-HER2 light chain
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
SEQ ID NO: 18 - Anti-HER2 heavy chain
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDK
SEQ ID NO: 19 - Lumretuzumab heavy chain
QVQLVQSGAE VKKPGASVKV SCKASGYTFR SSYISWVRQA PGQGLEWMGW IYAGTGSPSY
NQKLQGRVTM TTDTSTSTAY MELRSLRSDD TAVYYCARHR DYYSNSLTYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPG
SEQ ID NO: 20 - Lumretuzumab light chain
DIVMTQSPDS LAVSLGERAT INCKSSQSVL NSGNQKNYLT WYQQKPGQPP KLLIYWASTR
ESGVPDRFSG SGSGTDFTLT ISSLQAEDVA VYYCQSDYSY PYTFGQGTKL EIKRTVAAPS
VFIFPPSDEQ LKSGTASVVC LLNNFYPREA KVQWKVDNAL QSGNSQESVT EQDSKDSTYS
LSSTLTLSKA DYEKHKVYAC EVTHQGLSSP VTKSFNRGEC
SEQ ID NO: 21 - Seribantumab heavy chain
EVQLLESGGG LVQPGGSLRL SCAASGFTFS HYVMAWVRQA PGKGLEWVSS ISSSGGWTLY
ADSVKGRFTI SRDNSKNTLY LQMNSLRAED TAVYYCTRGL KMATIFDYWG QGTLVTVSSA
STKGPSVFPL APCSRSTSES TAALGCLVKD YFPEPVTVSW NSGALTSGVH TFPAVLQSSG
LYSLSSVVTV PSSNFGTQTY TCNVDHKPSN TKVDKTVERK CCVECPPCPA PPVAGPSVFL
FPPKPKDTLM ISRTPEVTCV VVDVSHEDPE VQFNWYVDGV EVHNAKTKPR EEQFNSTFRV
VSVLTVVHQD WLNGKEYKCK VSNKGLPAPI EKTISKTKGQ PREPQVYTLP PSREEMTKNQ
VSLTCLVKGF YPSDIAVEWE SNGQPENNYK TTPPMLDSDG SFFLYSKLTV DKSRWQQGNV
FSCSVMHEAL HNHYTQKSLS LSPGK
SEQ ID NO: 22 - Seribantumab light chain
QSALTQPASV SGSPGQSITI SCTGTSSDVG SYNVVSWYQQ HPGKAPKLII YEVSQRPSGV
SNRFSGSKSG NTASLTISGL QTEDEADYYC CSYAGSSIFV IFGGGTKVTV LGQPKAAPSV
TLFPPSSEEL QANKATLVCL VSDFYPGAVT VAWKADGSPV KVGVETTKPS KQSNNKYAAS
SYLSLTPEQW KSHRSYSCRV THEGSTVEKT VAPAECS
SEQ ID NO: 23 - KTN3379 light chain
QSVLTQPPSA SGTPGQRVTI SCSGSLSNIG LNYVSWYQQL PGTAPKLLIS RNNQRPSGVP
DRFSGSKSGT SASLAISGLR SEDEADYYCA AWDDSPPGEA FGGGTKLTVL GQPKAAPSVT
LFPPSSEELQ ANKATLVCLI SDFYPGAVTV AWKADSSPVK AGVETTKPSK QSNNKYAASS
YLSLTPEQWK SHRSYSCQVT HEGSTVEKTV APTECS
SEQ ID NO: 24 - Patritumab Subunit 1
QVQLQQWGAG LLKPSETLSL TCAVYGGSFS GYYWSWIRQP PGKGLEWIGE INHSGSTNYN
PSLKSRVTIS VETSKNQFSL KLSSVTAADT AVYYCARDKW TWYFDLWGRG TLVTVSSAST
KGPSVFPLAP SSKSTSGGTA ALGCLVKDYF PEPVTVSWNS GALTSGVHTF PAVLQSSGLY
SLSSVVTVPS SSLGTQTYIC NVNHKPSNTK VDKRVEPKSC DKTHTCPPCP APELLGGPSV
FLFPPKPKDT LMISRTPEVT CVVVDVSHED PEVKFNWYVD GVEVHNAKTK PREEQYNSTY
RVVSVLTVLH QDWLNGKEYK CKVSNKALPA PIEKTISKAK GQPREPQVYT LPPSREEMTK
NQVSLTCLVK GFYPSDIAVE WESNGQPENN YKTTPPVLDS DGSFFLYSKL TVDKSRWQQG
NVFSCSVMHE ALHNHYTQKS LSLSPGK
SEQ ID NO: 25 - Patritumab Subunit 2
QVQLQQWGAG LLKPSETLSL TCAVYGGSFS GYYWSWIRQP PGKGLEWIGE INHSGSTNYN
PSLKSRVTIS VETSKNQFSL KLSSVTAADT AVYYCARDKW TWYFDLWGRG TLVTVSSAST
KGPSVFPLAP SSKSTSGGTA ALGCLVKDYF PEPVTVSWNS GALTSGVHTF PAVLQSSGLY
SLSSVVTVPS SSLGTQTYIC NVNHKPSNTK VDKRVEPKSC DKTHTCPPCP APELLGGPSV
FLFPPKPKDT LMISRTPEVT CVVVDVSHED PEVKFNWYVD GVEVHNAKTK PREEQYNSTY
RVVSVLTVLH QDWLNGKEYK CKVSNKALPA PIEKTISKAK GQPREPQVYT LPPSREEMTK
NQVSLTCLVK GFYPSDIAVE WESNGQPENN YKTTPPVLDS DGSFFLYSKL TVDKSRWQQG
NVFSCSVMHE ALHNHYTQKS LSLSPGK
SEQ ID NO: 26 - Patritumab Subunit 3
DIEMTQSPDS LAVSLGERAT INCRSSQSVL YSSSNRNYLA WYQQNPGQPP KLLIYWASTR
ESGVPDRFSG SGSGTDFTLT ISSLQAEDVA VYYCQQYYST PRTFGQGTKV EIKRTVAAPS
VFIFPPSDEQ LKSGTASVVC LLNNFYPREA KVQWKVDNAL QSGNSQESVT EQDSKDSTYS
LSSTLTLSKA DYEKHKVYAC EVTHQGLSSP VTKSFNRGEC
SEQ ID NO: 27 - Patritumab Subunit 4
DIEMTQSPDS LAVSLGERAT INCRSSQSVL YSSSNRNYLA WYQQNPGQPP KLLIYWASTR
ESGVPDRFSG SGSGTDFTLT ISSLQAEDVA VYYCQQYYST PRTFGQGTKV EIKRTVAAPS
VFIFPPSDEQ LKSGTASVVC LLNNFYPREA KVQWKVDNAL QSGNSQESVT EQDSKDSTYS
LSSTLTLSKA DYEKHKVYAC EVTHQGLSSP VTKSFNRGEC
SEQ ID NO: 28 - Trastuzumab-based TriKE amino acid sequence
QVQLVESGGG LVQPGGSLRL SCAASGLTFS SYNMGWFRQA PGQGLEAVAS ITWSGRDTFY
ADSVKGRFTI SRDNSKNTLY LQMNSLRAED TAVYYCAANP WPVAAPRSGT YWGQGTLVTV
SSSGGGGSGG GGSGGGGSGG GGSGNWVNVI SDLKKIEDLI QSMHIDATLY TESDVHPSCK
VTAMKCFLLE LQVISLESGD ASIHDTVENL IILANNSLSS NGNVTESGCK ECEELEEKNI
KEFLQSFVHI VQMFINTSGS TSGSGKPGSG EGSTKGEVQL VESGGGLVQP GGSLRLSCAA
SGFNIKDTYI HWVRQAPGKG LEWVARIYPT NGYTRYADSV KGRFTISADT SKNTAYLQMN
SLRAEDTAVY YCSRWGGDGF YAMDYWGQGT LVTVSSGSTS GSGKPGSGEG STKGDIQMTQ
SPSSLSASVG DRVTITCRAS QDVNTAVAWY QQKPGKAPKL LIYSASFLYS GVPSRFSGSR
SGTDFTLTIS SLQPEDFATY YCQQHYTTPP TFGQGTKVEI KRTV
SEQ ID NO: 29 - Trastuzumab-based TriKE DNA sequence - Human codon optimized
CAGGTGCAGC TGGTGGAGTC TGGGGGAGGC TTGGTGCAGC CTGGGGGCTC TCTGAGACTC
TCCTGTGCAG CCTCTGGCCT CACCTTCAGT AGCTATAACA TGGGCTGGTT CCGCCAGGCT
CCAGGGCAAG GCCTTGAGGC TGTAGCATCT ATTACCTGGA GTGGTCGGGA CACATTCTAT
GCAGACTCCG TGAAGGGCCG ATTCACCATC TCCAGAGACA ACTCCAAGAA CACTCTCTAT
CTGCAAATGA ACAGCCTGCG CGCGGAGGAC ACGGCCGTTT ATTATTGTGC TGCAAACCCC
TGGCCAGTGG CGGCGCCACG TAGTGGCACC TACTGGGGCC AAGGGACCCT GGTCACCGTC
TCCTCATCTG GCGGCGGCGG TTCTGGTGGA GGAGGTAGTG GGGGGGGAGG AAGCGGAGGG
GGTGGCTCAG GGAACTGGGT GAATGTAATA AGTGATTTGA AAAAAATTGA AGATCTTATT
CAATCTATGC ATATTGATGC TACTTTATAT ACGGAAAGTG ATGTTCACCC CAGTTGCAAA
GTAACAGCAA TGAAGTGCTT TCTCTTGGAG TTACAAGTTA TTTCACTTGA GTCCGGAGAT
GCAAGTATTC ATGATACAGT AGAAAATCTG ATCATCCTAG CAAACAACAG TTTGTCTTCT
AATGGGAATG TAACAGAATC TGGATGCAAA GAATGTGAGG AACTGGAGGA AAAAAATATT
AAAGAATTTT TGCAGAGTTT TGTACATATT GTCCAAATGT TCATCAACAC TTCTGGCAGT
ACCAGCGGGT CAGGGAAACC TGGCAGTGGG GAAGGTTCCA CAAAAGGTGA GGTTCAGCTC
GTGGAATCCG GCGGCGGGCT GGTCCAACCA GGTGGGAGTC TCCGCCTGTC ATGTGCCGCA
AGCGGATTCA ATATAAAAGA TACATATATA CATTGGGTAA GACAGGCCCC CGGTAAGGGT
CTGGAGTGGG TTGCCAGAAT TTATCCCACT AATGGATACA CTCGTTACGC CGATTCTGTG
AAAGGCCGGT TTACCATCTC TGCTGACACC TCAAAGAACA CCGCGTACCT CCAGATGAAC
TCCCTGAGGG CAGAGGACAC GGCTGTCTAT TATTGCTCTC GCTGGGGCGG CGACGGGTTT
TATGCCATGG ATTACTGGGG TCAGGGGACC CTAGTTACAG TGAGCAGCGG TAGTACTTCT
GGGAGCGGCA AGCCTGGCTC CGGAGAAGGA TCCACCAAAG GGGATATCCA GATGACGCAG
AGCCCATCAT CATTGAGTGC CAGTGTGGGC GACCGGGTCA CGATCACCTG TAGGGCATCT
CAGGACGTAA ACACAGCGGT GGCATGGTAC CAGCAAAAAC CTGGAAAGGC CCCAAAACTT
TTGATCTACA GCGCTAGCTT CTTATACTCC GGCGTCCCCT CACGATTCTC CGGCTCCAGA
AGTGGAACAG ACTTTACTCT GACAATTTCT TCGCTTCAGC CCGAGGATTT TGCTACCTAT
TATTGCCAGC AACACTACAC AACCCCTCCG ACTTTCGGAC AAGGGACAAA GGTGGAAATT
AAGAGGACTG TG
SEQ ID NO: 30 - Trastuzumab-based TriKE DNA sequence - E. coli codon optimized
CAGGTGCAGC TGGTGGAGTC TGGTGGCGGC TTGGTGCAGC CTGGTGGCTC TCTGCGCCTG
TCCTGTGCGG CCTCTGGCCT CACCTTCAGC AGCTATAACA TGGGCTGGTT CCGCCAGGCT
CCAGGACAAG GCCTTGAGGC TGTGGCGTCT ATTACCTGGA GTGGTCGGGA CACCTTCTAT
GCGGACTCCG TGAAAGGCCG TTTCACCATC TCGCGTGACA ATTCCAAGAA CACGCTGTAT
CTGCAAATGA ACAGCCTGCG CGCGGAGGAC ACGGCCGTTT ATTATTGTGC GGCAAACCCC
TGGCCGGTTG CGGCGCCGCG TAGTGGCACC TACTGGGGCC AAGGGACCCT GGTCACCGTC
TCCTCATCTG GCGGCGGCGG TAGCGGTGGC GGAGGTAGCG GGGGGGGTGG AAGCGGTGGT
GGTGGCTCAG GGAACTGGGT GAATGTAATA AGTGATTTGA AAAAAATTGA AGATCTGATT
CAGAGCATGC ATATTGATGC GACGTTATAT ACGGAATCGG ATGTTCACCC AAGTTGCAAA
GTTACAGCAA TGAAATGCTT TTTGTTAGAG TTACAAGTTA TTTCACTTGA GTCGGGAGAT
GCAAGTATTC ATGATACTGT AGAAAATCTG ATCATCCTGG CAAACAACAG CTTGTCGTCG
AATGGGAATG TAACAGAATC TGGATGCAAA GAATGTGAAG AACTGGAAGA AAAAAATATT
AAAGAATTTT TGCAGAGTTT TGTTCACATT GTCCAAATGT TCATCAACAC TTCTGGCAGT
ACCAGCGGTT CAGGTAAACC GGGCAGCGGG GAAGGTTCCA CAAAAGGTGA AGTTCAGCTC
GTGGAAAGCG GCGGCGGTCT GGTCCAGCCA GGTGGGAGTC TCCGCCTGTC ATGTGCCGCG
AGCGGTTTTA ATATCAAAGA TACATATATA CATTGGGTAA GACAGGCCCC GGGTAAGGGT
CTGGAGTGGG TTGCGCGTAT TTATCCGACG AATGGATACA CTCGTTACGC CGATTCTGTG
AAAGGCCGCT TTACCATCTC AGCCGACACC TCAAAGAACA CCGCGTACTT ACAGATGAAC
TCCCTGCGCG CAGAAGACAC GGCTGTCTAT TATTGCTCGC GCTGGGGCGG CGACGGTTTT
TATGCCATGG ATTACTGGGG TCAGGGGACC CTAGTTACTG TGAGCAGCGG TAGTACTTCT
GGGAGCGGCA AACCTGGCTC CGGAGAAGGT TCGACCAAAG GGGATATCCA GATGACGCAG
AGCCCGTCAT CAGTGTGGGC CAGTGTGGGC GATCGGGTCA CGATCACCTG CCGTGCATCG
CAGGATGTAA ATACAGCGGT GGCATGGTAC CAGCAAAAAC CTGGAAAGGC CCCAAAACTT
CTGATCTACA GCGCTAGCTT CTTATACTCC GGCGTCCCGT CACGATTTTC CGGCTCCCGT
AGTGGAACGG ACTTTACTCT GACAATTTCT TCGCTTCAGC CCGAGGATTT TGCTACCTAT
TATTGCCAGC AACACTACAC CACCCCGCCG ACTTTCGGCC AAGGGACGAA AGTGGAAATT
AAGAGGACGG TG
SEQ ID NO: 31 - Signal peptide/Anti-HER2 light chain/linker/wtIL15/linker/cam16
MGWSCIILFL VATATGVHSD IQMTQSPSSL SASVGDRVTI TCRASQDVNT AVAWYQQKPG
KAPKLLIYSA SFLYSGVPSR FSGSRSGTDF TLTISSLQPE DFATYYCQQH YTTPPTFGQG
TKVETKRTVA APSVFIFPPS DEQLKSGTAS VVCLLNNFYP REAKVQWKVD NALQSGNSQE
SVTEQDSKDS TYSLSSTLTL SKADYEKHKV YACEVTHQGL SSPVTKSFNR GECGGGGSGG
GGSNWVNVIS DLKKIEDLIQ SMHIDATLYT ESDVHPSCKV TAMKCFLLEL QVISLESGDA
SIHDTVENLI ILANNSLSSN GNVTESGCKE CEELEEKNIK EFLQSFVHIV QMFINTSGST
SGSGKPGSGE GSTKGQVQLV ESGGGLVQPG GSLRLSCAAS GLTFSSYNMG WFRQAPGQGL
EAVASITWSG RDTFYADSVK GRFTISRDNS KNTLYLQMNS LRAEDTAVYY CAANPWPVAA
PRSGTYWGQG TLVTVSS
SEQ ID NO: 32 - Signal peptide/Anti-HER2 heavy chain
MGWSCIILFL VATATGVHSE VQLVESGGGL VQPGGSLRLS CAASGFNIKD TYIHWVRQAP
GKGLEWVARI YPTNGYTRYA DSVKGRFTIS ADTSKNTAYL QMNSLRAEDT AVYYCSRWGG
DGFYAMDYWG QGTLVTVSSA STKGPSVFPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDK
SEQ ID NO: 33 - Signal peptide/Anti-HER2 heavy chain Fab/T2A/signal peptide/Anti-HER2
light chain/linker/wtIL15/linker/cam16
MGWSCIILFL VATATGVHSE VQLVESGGGL VQPGGSLRLS CAASGFNIKD TYIHWVRQAP
GKGLEWVARI YPTNGYTRYA DSVKGRFTIS ADTSKNTAYL QMNSLRAEDT AVYYCSRWGG
DGFYAMDYWG QGTLVTVSSA STKGPSVFPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKEGRGSL LTCGDVEENP GPMGWSCIIL FLVATATGVH SDIQMTQSPS SLSASVGDRV
TITCRASQDV NTAVAWYQQK PGKAPKLLIY SASFLYSGVP SRFSGSRSGT DFTLTISSLQ
PEDFATYYCQ QHYTTPPTFG QGTKVEIKRT VAAPSVFIFP PSDEQLKSGT ASVVCLLNNF
YPREAKVQWK VDNALQSGNS QESVTEQDSK DSTYSLSSTL TLSKADYEKH KVYACEVTHQ
GLSSPVTKSF NRGECGGGGS GGGGSNWVNV ISDLKKIEDL IQSMHIDATL YTESDVHPSC
KVTAMKCFLL ELQVISLESG DASIHDTVEN LIILANNSLS SNGNVTESGC KECEELEEKN
IKEFLQSFVH IVQMFINTSG STSGSGKPGS GEGSTKGQVQ LVESGGGLVQ PGGSLRLSCA
ASGLTFSSYN MGWFRQAPGQ GLEAVASITW SGRDTFYADS VKGRFTISRD NSKNTLYLQM
NSLRAEDTAV YYCAANPWPV AAPRSGTYWG QGTLVTVSS
SEQ ID NO: 34 - Linker
SGGGGSGGGG SGGGGSGGGG SG
SEQ ID NO: 35 - Linker
GSTSGSGKPG SGEGSTKG
SEQ ID NO: 36 - Signal peptide
MGWSCIILFL VATATGVHS
SEQ ID NO: 37 - T2A self-cleaving peptide
EGRGSLLTCG DVEENPGP

1. A compound comprising:
an NK cell engaging domain comprising a moiety that selectively binds to CD16;
an NK activating domain operably linked to the NK cell engaging domain comprising IL-15 or a functional fragment thereof, and
a targeting domain that selectively binds to HER2, HER3, or a HER2/IER3 heterodimer complex and is operably linked to the NK activating domain and the NK cell engaging domain.

2. The compound of claim 1, wherein the CD16 comprises CD16a.

3. The compound of claim 1, wherein the NK cell engaging domain comprises the amino acid sequence of SEQ ID NO:2.

4. The compound of claim 1, wherein the NK cell engaging domain moiety comprises an antibody or a binding fragment thereof.

5. The compound of claim 4, wherein the antibody or a binding fragment thereof is human, humanized, or camelid.

6. The compound of claim 1, wherein the IL-15 comprises the amino acid sequence of SEQ ID NO:4 or a functional variant thereof.

7. The compound of claim 6, wherein the functional variant of IL-15 comprises an N72D or N72A amino acid substitution as compared to SEQ ID NO:4.

8. The compound of claim 1, wherein the targeting domain comprises an antibody or a binding fragment thereof.

9. The compound of claim 8, wherein the antibody binding fragment comprises an scFv, a F(ab)2, a Fab, or a single-domain antibody fragment.

10. The compound of claim 8, wherein the targeting domain comprises trastuzumab, e23, lumretuzumab, seribantumab, KTN3379/CDX-3379, patritumab, elgemtumab, U3-1402, AV-203, GSK2849330, MM-111, MCLA-128, istiratumab, duligotumab, pertuzumab, or a functional variant thereof.

11. The compound of claim 8, wherein the targeting domain comprises the amino acid sequence of SEQ ID NO:6, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27.

12. The compound of claim 1, further comprising at least one flanking sequence linking two of the domains.

13. The compound of claim 12, further comprising a second flanking sequence linking the two linked domains with the third domain.

14. The compound of claim 13, wherein the flanking sequences flank the NK activating domain.

15. The compound of claim 13, wherein a first flanking sequence is C-terminal to the NK cell engaging domain and wherein a second flanking sequence is N-terminal to the anti-tumor targeting domain.

16. The compound of claim 1, further comprising a second targeting domain.

17. The compound of claim 1, further comprising a second NK cell engaging domain.

18. The compound of claim 1, further comprising a second NK activating domain.

19. A composition comprising:
the compound of claim 1; and
a pharmaceutically acceptable carrier.

20. The composition of claim 19, further comprising an additional therapeutic agent.

21. The composition of claim 20, wherein the additional therapeutic agent comprises a therapeutic agent that targets HER2, HER3, or the HER2/HER3 heterodimer complex.

22. A method comprising:
administering to a subject the compound of claim 1 in an amount effective to induce NK-mediated killing of a cancer cell.

23. A method for stimulating expansion of NK cells in vivo, the method comprising:
administering to a subject an amount of the compound of claim 1 effective to stimulate expansion of NK cells in the subject.

24. A method of treating cancer in a subject, the method comprising:
administering to the subject an amount of the compound of claim 1 effective for treating the cancer.

25. The method of claim 24, further comprising administering the compound prior to, simultaneously with, or following chemotherapy, surgical resection of a tumor, or radiation therapy.

26. The method of claim 23, wherein the chemotherapy comprises altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, or vinorelbine.

27. A method comprising:
administering to a subject the composition of claim 19 in an amount effective to induce NK-mediated killing of a cancer cell.

28. A method for stimulating expansion of NK cells in vivo, the method comprising:
administering to a subject an amount of the composition of claim 19 effective to stimulate expansion of NK cells in the subject.

29. A method of treating cancer in a subject, the method comprising:
administering to the subject an amount of the composition of claim 19 effective for treating the cancer.

30. The method of claim 29, further comprising administering the composition prior to, simultaneously with, or following chemotherapy, immunotherapy, surgical resection of a tumor, or radiation therapy.

31. The method of claim 30, wherein the chemotherapy comprises altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, or vinorelbine.

32. The method of claim 30, wherein the immunotherapy targets HER2, HER3, or the HER2/HER3 heterodimer.
